(19) United States
(12) Patent Application Publication
(10) Pub. No.: US 2003/0229363 A1
(43) Pub. Date: Dec. 11, 2003

(54) Methods and devices for facilitating the formation of connections between tissue structures

(76) Inventors: A. Adam Sharkawy, Union City, CA (US); Florian Ludwig, Eisingen (DE)

Correspondence Address: BOZICEVIC, FIELD & FRANCIS LLP, 200 MIDDLEFIELD RD, SUITE 200, MENLO PARK, CA, 94025, US

(21) Appl. No.: 10/163,794

(22) Filed: Jun. 5, 2002

(51) Int. Cl. A61B 17/08

(52) U.S. Cl. 606/153; 623/1.23; 623/1.44

(57) ABSTRACT

Methods and devices are provided to facilitate the natural formation of a connection between tissue structures within the body. Certain of the subject methods provide for the formation on an anastomotic site between a graft vessel and a native vessel, such as vessels of the cardiovasculature, peripheral vasculature and neurovasculature, angiogenic, by means of facilitating angiogenic and/or arteriogenic processes at one or more selected points of contact or close proximity between the vessels. The subject devices include a mechanism for positioning or situating one vessel adjacent to another vessel, in situ, wherein a selected portion of each vessel is in contact or in close proximity with the other vessel such that a natural bond is formed between the outer tissue surfaces of the vessels at the point of contact or close proximity followed by the naturally occurring angiogenic and/or arteriogenic processes of the body.

### FIELD OF THE INVENTION

The present invention is related to the formation of natural connections between tissue structures within the body by means of the body's own biological and physiological processes. More particularly, the invention is related to the formation of connections between vessels by means of the body's angiogenic and arteriogenic processes.

Relevant Literature

U.S. Patents of interest include: Arras, M., W. D. Ito, et al. (1998), “Monocyte activation in angiogenesis and collateral growth in the rabbit hind limb,” J Clin Invest 101(1): 40-50; U.S. Pat. No. 5,972,903 to Barron and Botvinick, “Method For Promoting Angiogenesis Using Heparin and Adenosine” (1999); Bigelow, W. G., H. E. Aldridge, et al. (1966), “Internal mammary implantation (Vineberg operation) for coronary heart disease: in angiography and long-term follow up,” Ann Surg 164(3): 457-64; Bombardini, T. and E. Picano (1997), “The coronary angiogenetic effect of heparin: experimental basis and clinical evidence,” Angiology 48(11): 969-76; Buschmann, I. and W. Schaper (2000), “The pathophysiology of the collateral circulation (arteriogenesis),” J Pathol 190(3): 338-42; Cai, W., R. Vosschulte, et al. (2000), “Altered balance between extracellular proteolysis and antiproteolysis is associated with adaptive coronary arteriogenesis [In Process Citation],” J Mol Cell Cardiol 32(6): 997-1011; Carmeliet, P. (1999), “Basic Concepts of (Myocardial) Angiogenesis: Role of Vascular Endothelial Growth Factor and Angiopoietin,” Curr Interv Cardiol Rep 1(4): 322-335; Carmeliet, P. (2000), “Mechanisms of angiogenesis and arteriogenesis,” Nature Med. 6: 389-395; Carmeliet, P., Y. S. Ng, et al. (1999), “Impaired myocardial angiogenesis and ischemic cardiomyopathy in mice lacking the vascular endothelial growth factor isoforms VEGF164 and VEGF188,” Nat Med 5(5): 495-502; U.S. Pat. No. 4,921,838 to Catsimpoolas, Griffith, et al., Angiogenic and Blood Perfusion Inducing Properties of Amphiphilic Compounds (1990); Chawla, P. S., M. H. Keelan, et al. (1999), “Angiogenesis for the treatment of vascular diseases,” Int Angiol 18(3): 185-92; U.S. Pat. No. 5,318,957 to Cid, Grant, et al., Method of Stimulating Angiogenesis (1994); Diaz-Flores, L., R. Gutierrez, et al. (1994), “Intense vascular sprouting from rat femoral vein induced by prostaglandins E1 and E2,” Anat Rec 238(1): 68-76; Effler, D. B., F. M. Sones, Jr., et al. (1965), “Myocardial revascularization by Vineberg's internal mammary artery implant. Evaluation of postoperative results,” J Thorac Cardiovasc Surg 50(4): 527-33; U.S. Pat. No. 5,840,693 to Eriksson, Olofsson, et al., “Vascular Endothelial Growth Factor-B,” (1996); U.S. Pat. No. 5,928,939 to Eriksson, Olofsson, et al., “Vascular Endothelial Growth Factor-B and DNA Coding Therefor,” (1999); U.S. Pat. No. 6,214,800 to Fukiage, Azuma, et al., Angiogenesis Inhibitors (2001); U.S. Pat. No. 6,200,954 to Ge and Kini, “Small Peptides Having Potent Anti-Angiogenic Activity” 2001; Gowdak, L. H., L. Poliakova, et al. (2000), “Adenovirus-mediated VEGF(121) gene transfer stimulates angiogenesis in normoperfused skeletal muscle and preserves tissue perfusion after induction of ischemia,” Circulation 102(5): 565-71; Heil, M., M. Clauss, et al. (2000), “Vascular endothelial growth factor (VEGF) stimulates monocyte migration through endothelial monolayers via increased integrin expression,” Eur J Cell Biol 79(11): 850-7; U.S. Pat. No. 5,763,214 to Hu and Rosen ( ), “Fibroblast Growth Factor 11”, 1995; U.S. Pat. No. 5,932,540 to Hu, Rosen, et al., “Vascular Endothelial Growth Factor 2”, (1999); U.S. Pat. No. 6,040,157 to Hu, Rosen, et al., “Vascular Endothelial Growth Factor 2,” (2000); Kipshidze, N., P. Chawla, et al. (1999), “Fibrin meshwork as a carrier for delivery of angiogenic growth factors in patients with ischemic limb,” Mayo Clin Proc 74(8): 847-8; Kipshidze, N., V. Chekanov, et al. (2000), “Angiogenesis in a patient with ischemic limb induced by intramuscular injection of vascular endothelial growth factor and fibrin platform,” Tex Heart Inst J 27(2): 196-200; Laham, R. J., M. Post, et al. (2000), “Therapeutic Angiogenesis Using Local Perivascular and Pericardial Delivery,” Curr Interv Cardiol Rep 2(3): 213-217; Laham, R. J., M. Rezaee, et al. (2000), “Intrapericardial delivery of fibroblast growth factor-2 induces neovascularization in a porcine model of chronic myocardial ischemia,” J Pharmacol Exp Ther 292(2): 795-802; Laham, R. J., F. W. Sellke, et al. (1999), “Local perivascular delivery of basic fibroblast growth factor in patients undergoing coronary bypass surgery: results of a phase I randomized, double-blind, placebo-controlled trial,” Circulation 100(18): 1865-71; U.S. Pat. No. 6,200,259, “Method of Treating Cardiovascular Disease by Angiogenesis,” (2001); U.S. Pat. No. 4,895,838 to McCluer, Catsimpoolas, et al., “Method for Provoking Angiogenesis by Administration of Angiogenically Active Oligosaccharides,” (1990); Risau, W. (1994), “Angiogenesis and endothelial cell function,” Arzneimittelforschung 44(3A): 416-7; Risau, W. (1997), “Mechanisms of angiogenesis,” Nature 386(6626): 671-4; Schaper, W. (2000), “Quo vadis collateral blood flow? A commentary on a highly cited paper [comment],” Cardiovasc Res 45(1): 220-3; Schaper, W. and I. Buschmann (1999), “Arteriogenesis, the good and bad of it,” Cardiovasc Res 43(4): 835-7; Schaper, W. and W. D. Ito (1996), “Molecular mechanisms of coronary collateral vessel growth,” Circ Res 79(5): 911-9; Schaper, W., H. S. Sharma, et al. (1990), “Molecular biologic concepts of coronary anastomoses,” J Am Coll Cardiol 15(3): 513-8; Scholz, D., W. Ito, et al. (2000), “Ultrastructure and molecular histology of rabbit hind-limb collateral artery growth (arteriogenesis),” Virchows Arch 436(3): 257-70; Schumacher, B., T. Stegmann, et al. (1998), “The stimulation of neoangiogenesis in the ischemic human heart by the growth factor FGF: first clinical results,” J Cardiovasc Surg (Torino) 39(6): 783-9; Sellke, F. W., R. J. Laham, et al. (1998), “Therapeutic angiogenesis with basic fibroblast growth factor: technique and early results,” Ann Thorac Surg 65(6): 1540-4; Sellke, F. W., M. Tofukuji, et al. (1998), “Comparison of VEGF delivery techniques on collateral-dependent microvascular reactivity,” Microvasc Res 55(2): 175-8; Shrager, J. B. (1994), “The Vineberg procedure: the immediate forerunner of coronary artery bypass grafting [see comments],” Ann Thorac Surg 57(5): 1354-64; Trapp, W. G., J. D. Burton, et al. (1969), “Detailed anatomy of early Vineberg anastomosis,” J Thorac Cardiovasc Surg 57(3): 450-4; Unger, E. F., L. Goncalves, et al. (2000), “Effects of a single intracoronary injection of basic fibroblast growth factor in stable angina pectoris,” Am J Cardiol 85(12): 1414-9; Unger, E. F., C. D. Sheffield, et al. (1990), “Creation of anastomoses between an extracardiac artery and the coronary circulation. Proof that myocardial angiogenesis occurs and can provide nutritional blood flow to the myocardium,” Circulation 82(4): 1449-66; Unger, E. F., C. D. Sheffield, et al. (1991), “Heparin promotes the formation of extracardiac to coronary anastomoses in a canine model,” Am J Physiol 260(5 Pt 2): H1625-34; Vineberg, A. (1946), “Development of an anastomosis between the coronary vessels and a transplanted internal mammary artery,” Canadian Medical Association Journal 55: 117-119; Vineberg, A. (1949), “Development of Anastomosis between the Coronary Vessels and a Transplanted Internal Mammary Artery,” Journal of Thoracic Surgery 18: 839-850; Vineberg, A. (1975), “Evidence that revascularization by ventricular-internal mammary artery implants increases longevity. Twenty-four year, nine month follow-up,” J Thorac Cardiovasc Surg 70(3): 381-97; Vineberg A, A. S., Sahi S. (1975), “Direct revascularzation of acute myocardial infarction by implantation of left internal mammary artery into infarcted left ventricular myocardium,” Surg Gynecol Obstet 140: 44-52; Vineberg, A. and D. Miller (1953), “Functional Evaluation of an Internal Mammary Coronary Artery Anastomosis,” American Heart Journal 45: 873-888; Vineberg A. M., M. G. (1951), “Internal mammary coronary anastomosis in the surgical treatment of coronary artery insufficiency,” Can Med Assoc J 64: 204; U.S. Pat. No. 5,470,831 to Whitman, Wohl, et al., “Angiogenic Peptides,” (1995); Witzenbichler, B., T. Asahara, et al. (1998), “Vascular endothelial growth factor-C (VEGF-C/VEGF-2) promotes angiogenesis in the setting of tissue ischemia,” Am J Pathol 153(2): 381-94. Sharkawy et al (1998) “Engineering the tissue which encapsulates subcutaneous implants. II. Plasma-tissue exchange properties”, J Biomed Mater Res., 40(4):586-97; Sharkawy et al (1997), “Engineering the tissue which encapsulates subcutaneous implants. I. Diffusion properties,” J. Biomed Mater Res., 37(3):401-12.

### BACKGROUND OF THE INVENTION

Diseases of the cardiovascular and peripheral vascular systems affect millions of people each year. The cost of treating such diseases is enormous. A particularly prevalent form of vascular disease is a reduction in the blood supply leading to an area of tissue within the body, which creates a great risk for ischemia in that tissue area. Most commonly, such reduction in blood supply is caused by atherosclerosis.

In the context of the cardiovasculature, atherosclerosis can cause plaque to form in the coronary arteries thereby causing a partial blockage or complete occlusion of an artery and restricting blood flow to an area of the heart's myocardium which can lead to a myocardial infarction, i.e., a heart attack. In many cases, such a blockage or restriction in the blood flow leading to the heart can be treated by a surgical procedure known as a coronary artery bypass graft (CABG) procedure. In the CABG procedure, the surgeon typically dissects one end of a source artery proximate the heart, such as an internal mammary artery (IMA), or removes a portion of a free graft from another part of the body, typically the saphenous vein in the leg, to use as a graft vessel to bypass the obstruction in the affected coronary artery to restore normal blood flow to the heart. The graft vessel is connected to the obstructed vessel by means of an anastomosis procedure wherein the graft vessel is sutured to the obstructed vessel at an arteriotomy site made within the obstructed vessel.

The patency of the anastomosis is crucial to a successful bypass. Improperly anastomosed vessels may lead to leakage, create thrombus and/or lead to further stenosis at the connection site, possibly requiring re-operation and further increasing the risk of stroke. As such, forming the anastomosis is the most critical procedure in CABG surgery, requiring precision and accuracy on the part of the surgeon. The current gold standard for forming the anastomosis is by means of suturing; however, suturing has its disadvantages. In addition to the inherent inconsistencies in suture placement and stitch size and the lack of reproducibility, suturing an anastomosis can be very time consuming. Of course, the greater the number of vessels bypassed and, thus, the greater the number anastomoses required (i.e., pedicled arteries adjacent the heart may require only a single anastomosis to form a bypass, however, a wholly dissected vessel bypass will require at least two anastomoses to form a bypass), increases the overall procedure time. Additionally, hand suturing requires a fair amount of exposure at the bypass site which necessitates traumatic surgical incisions into the chest and manipulation of the ribs and subject organs.

Advances in anastomotic instruments have been devised in the attempt to provide greater reproducibility of a precise anastomosis, minimize the surgical incision size and the size of the surgical field, reduce the number of manipulations involved in the anastomotic procedure, and reduce the time that is required to complete an anastomosis. Many of these new instruments are stapling devices which deploy one or more staples at the anastomotic site in a single-motion action. Although stapling can save time, great precision and accuracy are required to ensure that the edges of the arteriotomy site of the native vessel and the edges of the dissected end (for an end-to-side anastomosis) or of the arteriotomy site (for a side-to-side anastomosis) of the graft vessel are properly everted and aligned prior to placement of the staples. An improperly placed staple can be very difficult to remove at the risk of tearing or damaging the tissue at the anastomosis site. Additionally, many of these instruments still require luminal access through the vessel which limits such anastomoses to single-ended free grafts.

Other technologies in the area of anastomosis are being developed with the hope of overcoming the disadvantages of suturing and stapling by simplifying the anastomosis procedure, and reducing the risk of improper adaptation between the vessels, the potential damage to the vessel tissue and the time necessary to complete an anastomosis. However, none of these devices are currently commercially available and may not be for some time to come.

Due to the accuracy required to form a patent anastomosis, conventional CABG surgery requires the patient to be placed on cardiopulmonary bypass, commonly known as the heart-lung bypass machine. Cardiopulmonary bypass involves stopping the heart by means of cardioplegic arrest and cross-clamping the aorta, at which point unoxygenated blood is removed from the patient's circulation, oxygenated and resupplied to the body. This allows the surgeon to operate on a motionless heart in a substantially bloodless surgical field which greatly facilitates precision and visibility during the anastomosis procedure. However, there are many risks associated with cardiopulmonary bypass, cardioplegic arrest and aortic cross-clamping which are commonly known by those skilled in the art of cardiac surgery. The most serious of these risks is the increase in the likelihood of stroke due to the need to cut through and clamp the aorta which procedures are likely to embolize plaque from the aortic wall. Additionally, patients who undergo surgeries using cardiopulmonary bypass often require extended hospital stays and experience lengthy recoveries. Thus, while conventional CABG surgery produces beneficial results for many patients, numerous others who might benefit from such surgery are unable or unwilling to undergo the trauma and risks of conventional procedures. Furthermore, cardiopulmonary bypass greatly increases the length and cost of a CABG surgery.

In recent years, less invasive surgical tools and techniques have been developed so that CABG procedures could be performed through smaller incisions and/or while the heart is beating. As a result, the surgery is much less invasive, the risks to the patient are minimized, and the time for recovery required by the patient and the cost of the procedure are significantly reduced. However, despite their advantages, beating-heart CABG procedures are not widely practiced, in part, because of the difficulty in performing the anastomosis procedure while the heart muscle is continuing to contract and pump blood. Much skill and practice may be required before a surgeon is fully competent to perform such beating-heart procedures. To date, few surgeons have adopted beating-heart CABG procedures.

There are still other less invasive, non-surgical procedures which have been developed as alternatives to or adjunct to CABG surgery. These include, for example, percutaneous transluminal coronary angioplasty (PTCA), atherectomy, stent placement and pharmacological treatments. The most common of these are PTCA and the use of stents which involve relatively short hospitalization stays and are relatively inexpensive. However, these benefits are mitigated by a significant restenosis rate. Similarly, the other alternatives suffer from their own drawbacks, and none of their outcomes are as effective and as long-lasting as those of CABG surgery.

Thus, there remains a need for methods and devices for treating ischemic tissue, in general, and for creating connections between vessels which overcome the disadvantages of prior art methods of interventional procedures, CABG surgery, cardiopulmonary bypass and conventional modes of making anastomoses, while providing outcomes and patency rates at least as good as CABG surgery. Moreover, it would be extremely advantageous to provide methods and devices for creating such connections without performing a surgical anastomosis.

### SUMMARY OF THE INVENTION

Methods, devices and systems are provided for facilitating, inducing or stimulating the natural formation of vascularized connections between tissue structures within the body. The subject methods, devices and systems accomplish such natural connections by means of facilitating the body's natural biological and physiological processes, and specifically the processes of angiogenesis and arteriogenesis.

Angiogenesis is the growth of new microvasculature, i.e., very small blood vessels, e.g., capillaries, in response to a stimulus that can include inflammation. Arteriogenesis is the expansion and remodeling of an existing artery or arteriole.

Thus, the present invention is directed to facilitating the creation of a natural anastomosis or tissue bond between two proximate conduits. Although many applications are contemplated, the present invention is particularly suited for facilitating angiogenic and arteriogenic responses between a blood-supplying vessel or tissue area and a blood-deprived vessel or tissue area in order to reestablish the perfusion of blood to the blood-deprived vessel and any adjacent ischemic tissue. For example, the present invention may be employed to facilitate the formation of such natural connections between an autologous or a synthetic graft vessel, including graft vessels fabricated in vitro or in situ, and a native vessel of the cardiovasculature, peripheral vasculature or neurovasculature. As such, the subject methods, devices and systems are very useful in the treatment of ischemic tissue and the prevention of ischemia of tissue deprived of oxygenated blood by establishing an adequate supply of blood to the target tissue area.

The natural process or processes for forming a natural tissue bond between tissue structures may be supported by the delivery of stimulants or tissue destabilizers to the points of contact or close proximity of the tissue structures. Alternatively or in addition to providing a natural connection between two vessels and the resulting blood perfusion there from, the present invention may further include an intervention such as the removal or disruption of tissue at the points of contact or close proximity between the tissue structures. The amount of tissue to be removed or disrupted may be as minimal as the removal of at least one cell layer of the epithelium from the external surfaces of one or more of the tissue structures, or may be as much as the disruption of the entire thickness of the tissue bond between the two tissue structures. The removal of epithelial cell layers may involve the use of laser, thermal, chemical or mechanical means. The disruption of the entire thickness of the tissue bond involves forming an opening in the tissue bond to create a fluid communication pathway between the two bonded tissue structures, e.g., an incision is made through the bonded tissue between two bonded vessel to allow blood to flow from the lumen of a blood-supplying vessel to the lumen of a blocked vessel or directly to the ischemic area deprived of blood by the vessel blockage.

The subject methods provide for the formation of a natural, vascularized tissue bond between two or more blood-carrying vessels at one or more selected locations at the ends or along the lengths thereof, by means of angiogenic and/or arteriogenic processes at one or more selected points of close proximity or contact between the tissue structures or vessels. In addition to facilitating naturally occurring angiogenic and arteriogenic responses (e.g., initiated by the inherent irritation that may occur by the contact between tissue surfaces), the subject methods may further facilitate such processes by providing an implantable device of the present invention for appositioning, adaptating or juxtaposing the vessels with respect to each other and/or by application of one or more various types of growth stimulants such as growth factors, proteins and and/or vessel wall destabilizers to the selected points of contact or close proximity between the vessels. The subject methods may further provide for the sustained delivery of such stimulants either by means of the implantable device (e.g., by topical application of the stimulants to the device, by integration of the stimulants into a biodegradable part of the device which sets free the stimulants upon elution or degradation), and/or by the administration of gene therapies.

The devices of the present invention may include a structure or mechanism for appositioning or situating one vessel or tissue structure adjacent to another vessel or tissue structure, in situ. More particularly, the subject devices place one or more selected portion of each vessel or tissue structure in physical contact or close proximity with the other vessel or structure such that a natural, vascularized tissue bond is formed between the outer tissue surfaces, between an end of a transected vessel and a tissue surface, or between ends of the vessels or structures at the one or more selected points of contact or close proximity by the naturally occurring angiogenic and/or arteriogenic processes of the body. These selected points of contact or close proximity may also serve as locations for the formation of fluid communication between the naturally bonded vessels.

The device may have a structure having a scaffold configuration or act as a scaffolding to hold the tissue surfaces in contact or in close proximity with each other. In other embodiments, the device is a substrate having a structure or surface morphology, such as a porous morphology, to provide angiogenic and/or artieriogenic stimulation and/or to conduct gene therapy at the points of contact or close proximity. In certain embodiments, the devices may further include a delivery system for the sustained release of agents to the areas of contact or close proximity between the tissue surfaces to stimulate these processes. Alternatively or additionally, the subject devices themselves may function as such a delivery system. Additionally, the subject devices or parts thereof may be biodegradable.

The systems of the present invention may include one or more of the subject devices and/or other instrumentation for surgically and/or percutaneously (by a catheter-based approach) accessing, presenting and selectively placing one or more vessels or tissue structures in close proximity to or in physical contact with each other. The subject systems may further include delivery devices or apparatus' for implanting the subject devices and/or sustained release systems and/or devices for conducting gene therapy to facilitate the body's angiogenic and arteriogenic processes in forming a vascularized tissue bond between the vessels. The subject systems may also include devices for establishing fluid communication at the bonded site between the naturally connected vessels.

Additionally, the systems of the present invention may include a “self-guiding” modality for facilitating the positioning or appositioning of one vessel or tissue structure with respect to another vessel or tissue structure. Such devices may include, for example, the incorporation of magnetic material in the subject devices and/or in the delivery devices used for positioning and appositioning one vessel or tissue structure adjacent to another vessel or tissue structure. The subject systems may further include instrumentation, e.g., a tissue cutting or piercing element, used to establish fluid communication between the naturally connected vessels. Such devices may be partially or completely integrated into the subject devices described in the previous paragraph and/or other devices used for positioning one vessel or tissue structure adjacent to another vessel or tissue structure.

For cardiovascular applications, certain such systems include instruments for providing a graft vessel, either an artery segment which has been freed from its native bed in close proximity to the heart (in situ), a pedicled artery in close proximity to the heart, a section of free graft vessel (e.g., a section of the saphenous vein or the radial artery) harvested from elsewhere in the body or a synthetic or in situ or in vitro fabricated graft, and instruments for placing such graft vessel in physical contact with or in close proximity to a targeted natural vessel or tissue structure deprived of oxygenated blood (e.g., a stenotic or occluded coronary artery) at one or more selected location proximate to the site of blood deprivation (e.g., distal to a stenosis or occlusion in the targeted vessel or at the site of ischemia caused by the deprivation).

Also provided by the present invention are kits which include at least one subject device and/or a subject system for practicing the methods of the present invention.

An advantage of the present invention is the elimination of sutures and other conventional anastomotic devices for creating connections between vessels in the body. Another advantage of the present invention is the obviation of a surgical connection between two vessels which further reduces the amount of skill, precision and accuracy required on behalf of a surgeon in order to form connections between vessels. Yet another advantage of the present invention is the ability to form such connections between small vessels and tissue structures through minimally invasive incisions. Another advantage is the ability to form such natural connections on a beating heart.

These and other features and advantages of the invention will become apparent to those persons skilled in the art upon reading the details of the methods, devices, systems and kits of the invention, as more filly described below.

### BRIEF DESCRIPTIONS OF THE DRAWINGS

FIGS. 1A-D illustrate certain method steps of the present invention for facilitating the formation of a natural connection between two vessels.

FIGS. 2A- and 2B illustrate other steps of another method of the present invention for facilitating both the natural and interventional establishment of a fluid communication between two vessels.

FIGS. 3A-C illustrate examples of the possible positioning between vessels for implementing the methods of FIGS. 1 and 2. In particular, FIG. 3A illustrates a possible position and location of a pedicled graft vessel with respect to a target native coronary artery; FIG. 3B illustrates the a possible positioning and location of a free graft vessel with respect to a target native coronary artery wherein the graft vessel supplies blood from the ascending aorta; and FIG. 3C illustrates a possible positioning and location of a graft vessel with respect to multiple targeted coronary arteries wherein the graft vessel supplies blood from the descending aorta.

FIGS. 4A and 4B are perspective views of an embodiment of a device of the present invention for appositioning vessels with respect to each other to facilitate the body's own angiogenic and/or arteriogenic processes.

FIG. 5A is a perspective view of another embodiment of an apposition device of the present invention. FIG. 5B is a cross-sectional view of the device of FIG. 5A.

FIG. 6A is a perspective view of another embodiment of an apposition device of the present invention. FIG. 6B is a cross-sectional view of the device of FIG. 6A.

FIG. 7A is a perspective view of another embodiment of an apposition device of the present invention. FIG. 7B is a cross-sectional view of the device of FIG. 7A.

FIG. 8A is a perspective view of yet another embodiment of an apposition device of the present invention. FIG. 8B is a cross-sectional view of the device of FIG. 8A.

FIG. 9 is a perspective view of a two-piece apposition device of the present invention.

FIGS. 10A and 10B illustrate perspective views of two other embodiments of an appositioning device of the present invention which utilize magnetic force to apposition tissue structures.

FIGS. 11A-D illustrate other embodiments of appositioning devices of the present invention providing a substrate material to facilitate the natural connection of the tissue structures which are appositioned.

FIG. 12 illustrates use of a system of the present invention for interventionally establishing fluid communication between two vessels which have been appositioned by means of the device of FIG. 10B.

FIGS. 13A-C illustrate use of another system of the present invention for endovascularly or intravascularly delivering and implanting the apposition device of FIG. 11A having a pre-attached graft vessel adjacent to a target tissue structure within the body wherein an intact vessel is used as a blood supply.

FIG. 14A illustrates a delivery approach for delivering and implanting the apposition device of FIG. 11B having a pre-attached graft vessel to a target tissue structure within the body wherein the left ventricle is used as a blood supply. FIG. 14B illustrates the apposition device of FIG. 11B delivered to a target site within the ventricular wall according to the delivery approach of FIG. 14A.

FIGS. 15 illustrates the apposition device of FIG. 11C having a graft vessel attached thereto and having been implanted by the delivery system of FIGS. 13A-C.

FIG. 16 illustrates the apposition device of FIG. 11D having a graft vessel attached thereto and having been implanted by the delivery system of FIGS. 13A-C.

### DETAILED DESCRIPTION OF THE INVENTION

Methods, devices and systems are provided for facilitating the natural formation or inducement of vascularized connections between tissue structures within the body, and are particularly useful for facilitating such natural connections between tissue vessels, such as blood vessels. More particularly, the subject methods, devices and systems accomplish such natural connections by means of facilitating the body's natural angiogenic and/or arteriogenic processes.

Before the present invention is further described, it is to be understood that this invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. It must also be noted that as used herein and in the appended claims, the singular forms “a”, “and”, and “the” include plural referents unless the context clearly dictates otherwise.

All publications mentioned herein are incorporated herein by reference to disclose and describe the methods and/or materials in connection with which the publications are cited. These publications are provided solely for their disclosure prior to the filing date of the present application. Nothing herein is to be construed as an admission that the present invention is not entitled to antedate such publication by virtue of prior invention. Further, the dates of publication provided on the respective publications may be different from the actual publication dates which may need to be independently verified.

The present invention will now be described in detail. A detailed description of the subject methods is first presented followed by a detailed description of the subject devices and systems, as well as a description of the subject kits. While the present invention is particularly suited for forming connections or junctures between the vessels of the cardiovasculature, peripheral vasculature and neurovasculature or between such vessels and other tissue structures and tissue surfaces, e.g., the myocardium, the present invention will primarily be described in the context of cardiovascular applications for the formation of vascularized connections between a stenosed blood coronary artery and a blood-supplying vessel (e.g., an internal mammary artery, or a harvested or synthetic free graft in fluid communication with a source of blood) to perfuse the ischemic tissue area deprived of blood by the stenosis and to prevent further ischemic damage. However, such exemplary application is not intended to limit the scope of the invention, and those skilled in the art will appreciate that the present invention is useful in other physiological applications.

Methods of the Present Invention

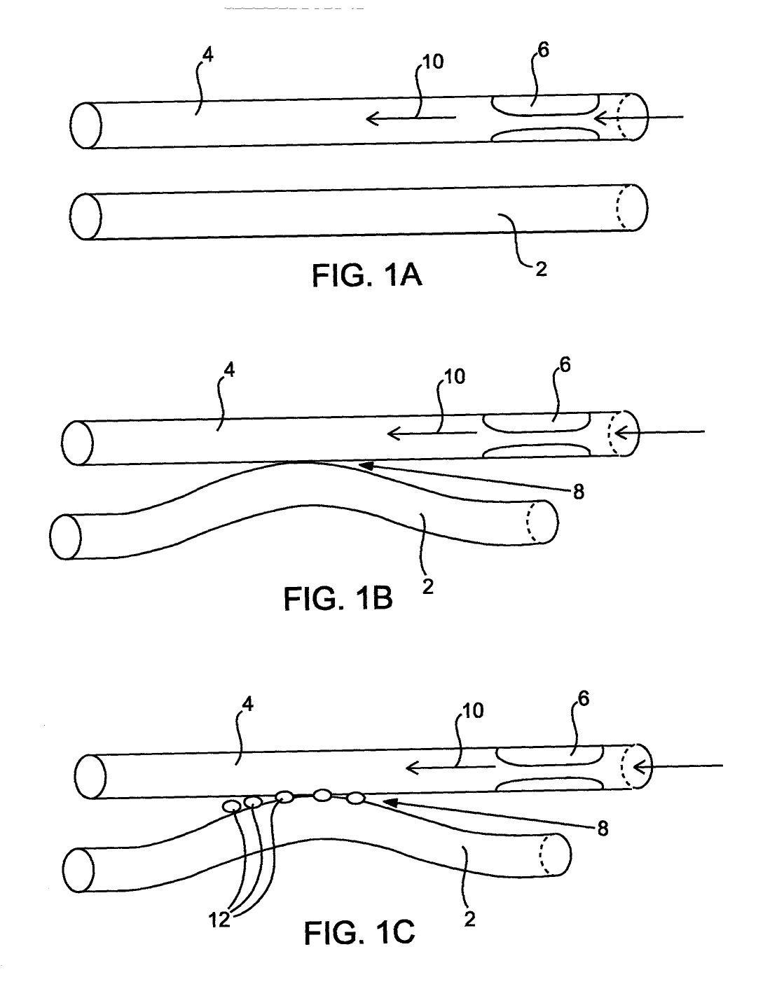

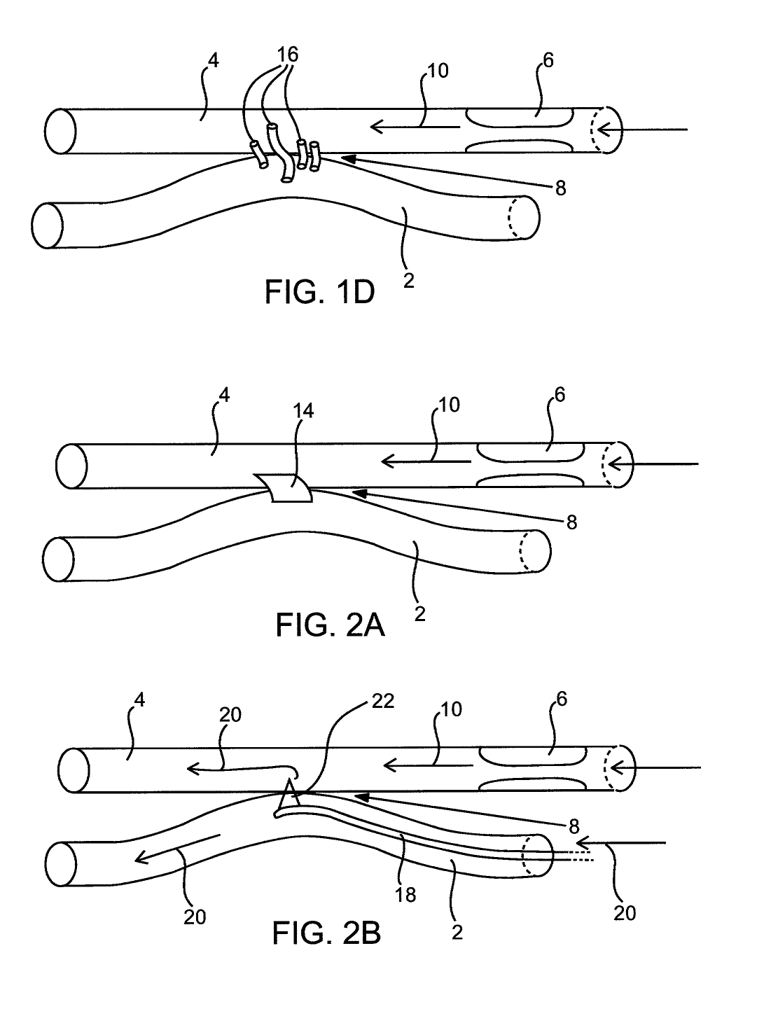

FIGS. 1A-D illustrate steps of a methods of the present invention for facilitating the formation of one or more natural connections between two tissue structures, and, in particular, for facilitating the formation of one or more natural connections between a graft vessel 2 and a stenosed coronary artery 4 (shown in FIG. 1A having a stenosis or blockage 6) or a stenosed, previously established graft vessel, or an ischemic area of tissue, each referenced interchangeably with the term “target tissue.” Stenosis in a blood-carrying vessel can restrict or completely block the flow of blood (such flow designated by arrow 10 in FIGS. 1A-D.).

First, initial access is made to the targeted area or area of interest, e.g., within the vicinity of the native coronary artery 4 to be bypassed or the area of myocardium to be perfused with blood, in which the two or more vessels are to be interconnected. With respect to performing the subject methods on the cardiovascular system, access can be made surgically through a sternotomy, mini-sternotomy, thoracotomy or mini-thoracotomy, or less invasively through a port provided within the chest cavity of the patient, e.g., between the ribs or in a subxyphoid area, with or without the visual assistance of an thorascope. Alternately, or in addition to a surgical approach wherein one or more steps of the procedure are performed endovascularly, access to the targeted surgical area may be provided by catheter-based instrumentation via percutaneous access with or without fluoroscopic and/or endoscopic assistance. Ultimately, the type and location of access is determined by the surgeon or interventionalist, e.g., a cardiologist, taking into consideration a given patient's particular physiological and medical indications.

An additional consideration is whether or not the patient is placed on cardiopulmonary bypass, i.e., whether the procedure is performed on a stopped or beating heart. As mentioned above, the relative ease with which the subject methods can be performed by those skilled in the art, allows the subject methods, in the context of cardiovascular procedures, to be performed on a beating heart.

After the initial access (i.e., surgical, percutaneous, port, etc.) is made to the area of interest, a suitable graft vessel or tissue structure 2 capable of supplying blood is selected and provided within the target area (see FIG. 1A). The graft vessel 2 may be a natural or prosthetic vessel or a hybrid vessel combining both natural and synthetic material within. Natural graft vessels may be autologous, allogenic or xenogenic vessels. Commonly used natural graft vessels include but are not limited to left and right internal mammary arteries, radial arteries, gastroepiploic arteries, sapphenous vein and femoro-popliteal arteries. The autologous graft vessels may be an in situ artery, a pedicled artery in close proximity to the targeted native vessel or a section of a vessel harvested from a part of the body remote from the targeted native vessel. For example, often when the native artery to be bypassed is the left anterior descending artery (LAD), the left internal mammary artery (LIMA) is used as the bypass vessel. In coronary artery bypass applications, harvested vessels most typically include saphenous veins and radial arteries.

Prosthetic grafts suitable for use with the present invention include those which are made of synthetic and/or pre-fabricated and/or, tissue engineered materials which are externally seeded with endothelium and/or are made of a nonthrombogenic material. Synthetic graft vessels typically include but are not limited to Dacron, expanded PTFE, carbonaceous materials, such as carbon fibers, silicone, polyurethane, polyglycolic acid, polylactic acid, and other similar biodegradable and nondegradable materials. Pre-fabricated, tissue engineered vessels are typically made of material which is collagenous in nature and include, but are not limited to, the pericardium, connective tissues, e.g., dura matter, tendons, ligaments, skin patches, mucosal patches, omentum, arteries, veins and the like, where the tissue is generally mammalian in nature, where specific species of interest include humans, cows, horses, pigs, sheep and primates.

Other prosthetic vessels suitable for use with the present invention are those which are formed or fabricated in situ from a pre-treated mandrel or scaffolding which is comprised in part of material treated to induce tissue growth thereon, such as disclosed in co-pending U.S. patent application Ser. No. 09/863,198, which is hereby incorporated by reference.

The step of providing such graft tissue structure 2, whether such graft tissue structure is a natural or pre-fabricated vessel or a vessel hosted in situ formed about and an implanted scaffold, mandrel or substrate positioned within proximity to the target vessel or tissue structure 4, may include one or more of the following: preparing a graft vessel from its natural tissue bed; dissecting or removing a graft vessel away from its natural tissue bed; transecting a graft vessel once (to create a pedicled end) or twice (to provide a section of free vessel completely detached from its natural setting); ligating such transected ends if appropriate, preparing the graft vessel, e.g., clipping off or ligating transected tributaries of the graft vessel; seeding or coating the graft vessel with selected agents including but not limited to angiogenic and/or arteriogenic growth factors, antiplatelets, anticoagulants and other proteins, stimulants, adhesives, etc.; loading a free graft vessel into a vessel delivery device, such as a delivery catheter, and delivering the free graft endovascularly or intravascularly to a target tissue structure, wherein the graft vessel may be or may not be preliminarily coupled to a device substrate for facilitating the formation of a natural connection between the graft vessel and the target tissue structure, etc.

The physician may also find it preferable to take additional steps to prepare one or more sections or surface areas of the targeted native vessel or tissue area 4, which may include seeding or coating native vessel 4 with selected agents such as but not limited to angiogenic and arteriogenic growth factors, antiplatelets, and anticoagulants and other proteins, stimulants, adhesives, etc. as described with respect to the graft vessel. The native vessel 4 may be further prepared by removing or clearing surface tissue, such as a very thin layer of myocardial tissue, or fat, from selected surface areas of vessel 4 to better expose such for contacting corresponding surface areas of the graft vessel 2 or to be able to position the graft vessel 2 in closer proximity to target vessel 4. The amount of tissue to be removed or disrupted may vary from the removal of at least one cell layer of the epithelium from the external surfaces of one or more of the tissue structure to the disruption of the entire thickness of the tissue bond between the two tissue structures. The removal of epithelial cell layers may involve the use of laser, thermal, chemical or mechanical means. The disruption of the entire thickness of the tissue bond involves forming an opening in the tissue bond to create a fluid communication pathway between the two bonded tissue structures, e.g., an incision is made through the bonded tissue between two bonded vessel lumens to allow blood to flow from a blood-supplying vessel to a blocked vessel or directly to the ischemic area deprived of blood by the vessel blockage.

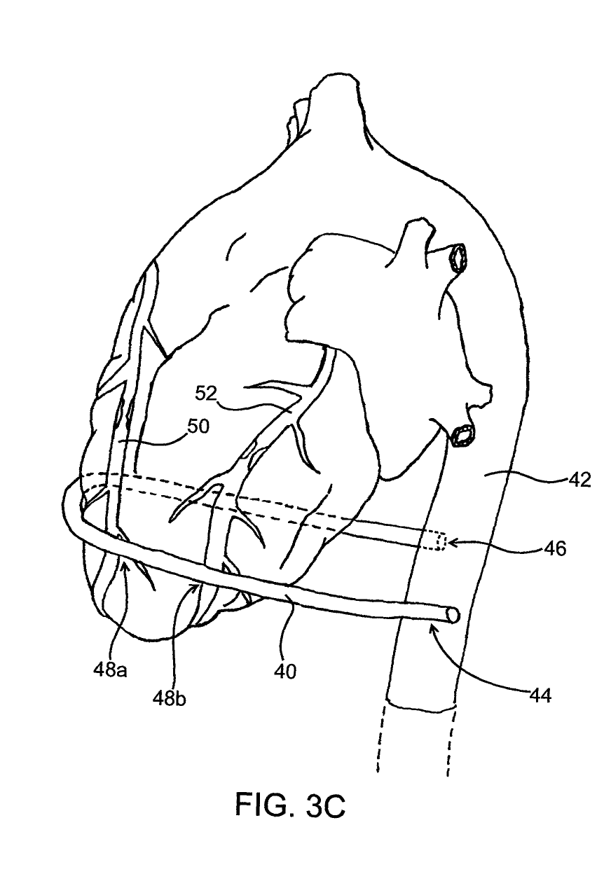

Next, as illustrated in FIG. 1B, graft vessel 2 is moved or placed adjacent to or appositioned or juxtaposed against target native vessel 4, such that at least one or more selected points of tissue or surface area of the graft vessel are caused to physically contact or reside in close proximity to at least one or more corresponding selected points 8 of the exposed surface area of the native vessel 4 or of the targeted tissue area. Preferably, a selected point of contact or close proximity 8 on the native vessel and/or tissue area is distal to or downstream of the stenosis or occlusion 6 within the native vessel 4 so as to supply blood to adjacent ischemic tissue and possibly prevent further ischemia. In certain embodiments, another selected point of contact or close proximity for the graft vessel is at a proximal portion or end (i.e., that portion or end that is to received blood upstream of the blocked vessel or of the ischemic tissue area) (not shown in the Figs.). The corresponding point of contact or close proximity may be at an upstream location on the target vessel itself or on another blood-supplying vessel such as the aorta or another arterial vessel (see e.g. FIG. 3B). The subject methods may further include additional points of contact or close proximity between the graft vessel and one or more target vessels, target tissue areas, or blood-supplying vessels. The points of contact or close proximity include any location of the subject structures, including a surface area or end portion, such as in an end-to-end or end-to-side vessel connection.

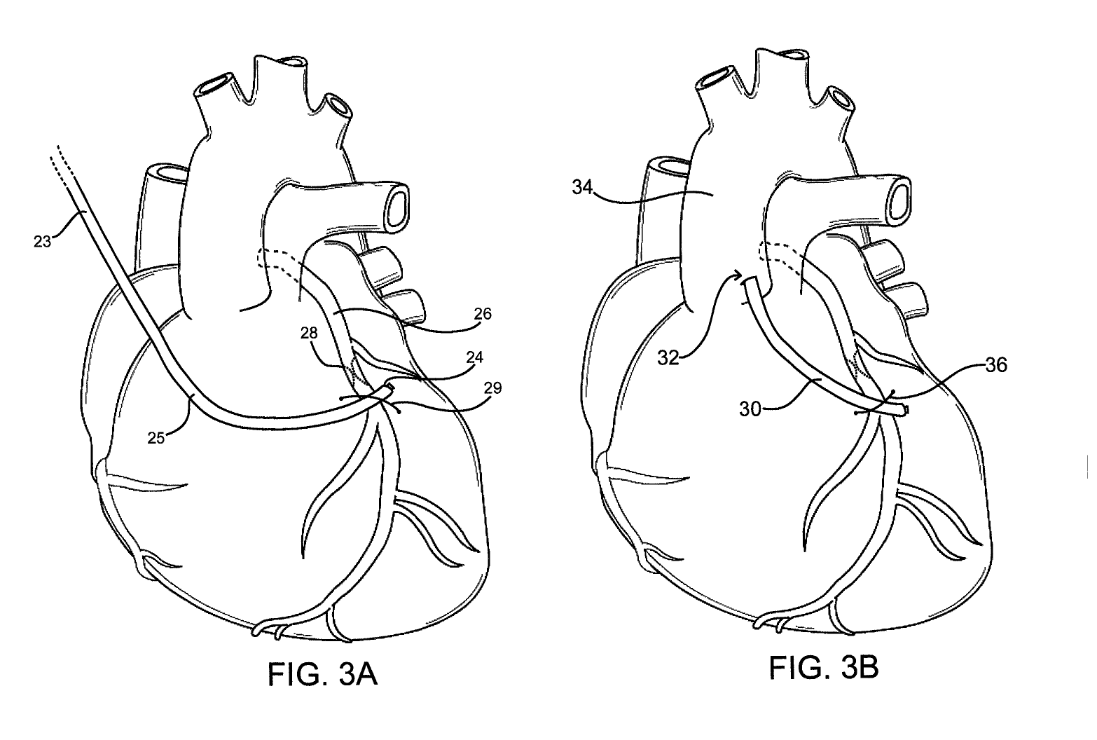

The position of the appositioned or juxtaposed vessels and/or tissue areas and the points of contact or close proximity 8 there between are then actively or substantially maintained at least until the body's angiogenic/arteriogenic processes forms a vascularized tissue bond between the contacting surface areas, bond sites or areas of close proximity between the graft and the target vessel, and fluid perfusion is established through the newly formed capillaries, arterioles and/or bigger arteries, or until an “anastomotic” site is formed by a tissue bond whereat fluid communication can be established by subsequent surgical intervention (the latter of which is described in greater detail below with respect to FIGS. 2A and 2B). Such substantial maintenance of the relative position of the graft vessel 2 prevents movement that is likely to occur due to the natural beating of the patient's heart.

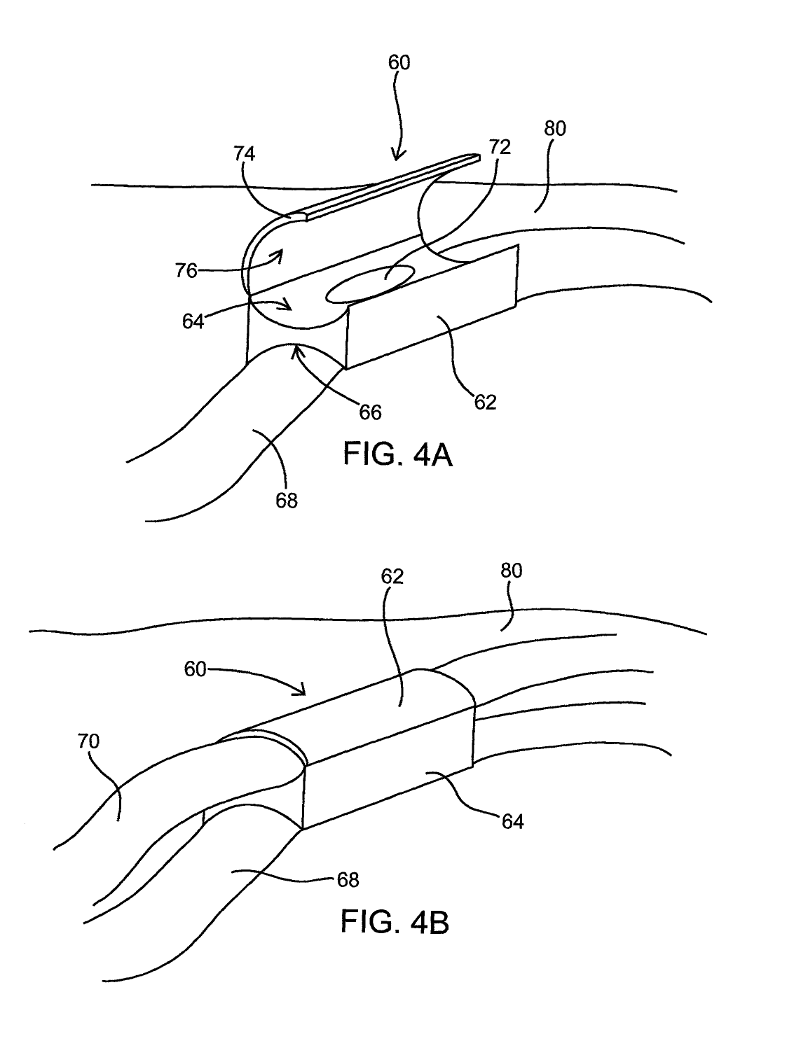

In certain of the subject methods, such contact or close proximity may be permanently maintained. In either case, the placement of and contact or close proximity between the tissue surface areas may be maintained by various means including but not limited to a biocompatible, implantable device (see FIGS. 4A, 4B, 5A, 5B, 6A, 6B, 7A, 7B, 8A, 8B, 9, 10A, 10B and 11A-D) particularly configured to hold the vessels or tissue surfaces in ideal apposition to and contact or close proximity with each other (discussed in greater detail in the section below entitled “Devices of the Present Invention”). Such biocompatible device may be may be subsequently removed from the patient after the points of contact or close proximity become angiogenically and arteriogenically bonded sites. The device may be configured to be easily taken apart or to be cut into smaller pieces in order to more easily remove the device from the bonded vessels. Access for the purpose of removing and/or cutting away the device is preferably accomplished less invasively, such as by port access or by a catheter-based approach. Alternatively, the device may be bioresorbable or biodegradable such that the device becomes absorbed or degrades at a rate that is sufficient to allow the angiogenic and arteriogenic processes to form capillaries and capillaries, arterioles and/or bigger arteries between the points of contact or close proximity.

Another technique of the subject methods for substantially maintaining the selected points of contact or close proximity include, for example, securing the graft vessel to the native vessel or tissue area by means of one or more sutures, such as sutures 29 and 36 of FIGS. 3A and 3B, respectively, attached to the epicardial surface of the myocardium. The points of contact or close proximity may be maintained without the use of a mechanical device, for example, as illustrated in FIG. 3C, by adequately tensioning the graft vessel 40 against the surface of the myocardium and/or by applying a biological adhesive to one or more contacting surface areas, either at or adjacent to the selected points of contact or close proximity 48a and 48b between the graft vessel 40 and one or more target native vessels 50 and 52, e.g., the LAD, the circumflex artery and/or the right coronary artery, etc.

The subject methods further involve supplying blood to or causing blood to be supplied to the graft vessel 2. In certain embodiments of the subject methods, the graft vessel may be provided having a pre-existing, natural communication with a supply of blood, e.g., in the case of pedicled arteries as illustrated in FIG. 3A or in the case of an in situ graft. In this embodiment, only a distal segment 24 of the graft vessel 25 will be freed and dissected from its native bed while a proximal end 23 is not dissected and remains intact with its natural blood supply, e.g., the left internal mammary artery. As such, graft vessel 25 also acts as the source or supply of blood. The transected and ligated distal end 24 is tied off and then relocated into close proximity or direct physical contact with the targeted native area or vessel 26, e.g., the left anterior descending artery, at a location distal to a blockage 28 within native vessel 26. In such embodiments, blood is supplied to the graft vessel prior to the placing, contacting or appositioning the graft vessel and native vessel(s) or target tissue area(s) in close proximity to each other.

In other embodiments of the subject methods, such blood supply to the graft vessel is otherwise established, as illustrated in FIGS. 3B and 3C, e.g., in the case of transected sections of natural vessels, i.e., free grafts, harvested from the patient or a donor, pre-fabricated sections of prosthetic vessels made partially or wholly of synthetic or tissue engineered material, and vessels which are formed in situ on implanted scaffolding. The establishment of a blood supply to such free graft vessels may be accomplished by establishing a connection between the graft vessel and the aorta or another arterial vessel or the left ventricle prior to, at or near the time of selectively placing such graft vessel with respect to the native vessel to be bypassed. For example, as shown in FIG. 3B, such methods may involve a single connection between a graft vessel 30 and a source of blood, such as ascending aorta 34, by means of proximal connection site 32. Alternatively, more than one such connection, as shown in FIG. 3C, such as proximal and distal connections 44, 46 between graft vessel 40 and a source of blood, such as the descending aorta 42, may be established. Alternatively, the source of blood may be a smaller artery such as the IMA wherein both ends of a free graft vessel are placed in fluid communication at two spaced apart locations along the length of the IMA so as to provide a “sling circuit” wherein the central part of the graft vessel is placed so as to be slung over or against the target tissue structure or area. The connections used to provide fluid communication between the graft vessel and the target tissue structure may be established by means of a conventional proximal anastomosis. Once the supply connection or connections are established, blood is allowed to flow into the lumen of the graft vessel and is thus subsequently suppliable to the native vessel or adjacent tissue area.

In other embodiments of the subject methods, such as in the case in which a graft vessel is formed or fabricated in situ from a pre-treated mandrel or scaffolding configured to induce tissue growth thereon, as disclosed in co-pending U.S. patent application Ser. No. 09/863,198, the establishment of such blood-supply communication to the graft vessel is necessarily performed subsequently, either days, weeks or months after placement of the mandrel or scaffolding at a target location. More specifically, the mandrel or scaffolding is positioned so as to have one or more points of contact or close proximity with one or more target vessels or tissue areas. As such, the morphology and/or porosity of the scaffolding facilitate angiogenic and/or arteriogenic responses to create a vessel structure about the scaffolding. After the graft vessel wall has been adequately formed about the mandrel or scaffolding, the mandrel or scaffolding is removed from the graft vessel and a blood supply is established to the newly-formed graft vessel, often by the aorta, which then facilitates arteriogenesis between the newly formed vessel and the target vessel or tissue area.

After or concurrent with establishing a blood supply to the graft vessel, the subject methods may further include the delivery of agents to the target area, and particularly to the selected points of contact or close proximity between the graft vessel and the target tissue or vessel, to stimulate or otherwise treat such points of contact or close proximity. More specifically, the agents are delivered to the selected points of contact or close proximity to stimulate these processes in order to establish fluid communication, i.e., blood flow, between the graft vessel and the target vessels or area. FIG. 1C illustrates agent 12 which has been applied or deposited at point of contact or close proximity 8 between graft vessel 2 and target vessel 4 in order to stimulate the angiogenic and arteriogenic processes to create the resulting capillaries and arterioles 16 (and sometimes larger arteries) between the adjoined points of contact or close proximity 8, as illustrated in FIG. 1D. Upon such response, fluid communication is established between graft vessel 2 and native vessel 4 distal to blockage 6, thereby perfusing the previously oxygen-deprived area with oxygenated blood.

Agents suitable for use with the present invention include but are not limited to stimulants and tissue destabilizers. Such tissue destabilizers are used to aid in breaking down the graft tissue and target target tissue structures, such as the vessel walls, to initiate the angiogenic and arteriogenic processes. Suitable tissue destabilizers for use with the present invention include but are not limited to proteases, such as collagenase and elastase. Such stimulants are used to enhance the body's inherent angiogenic/arteriogenic, and include but are not limited to cytokines, monocyte attractants and chemoattractants and/or gene therapy agents encoding such stimulants. Suitable cytokines include but are not limited to growth factors of the family of vascular endothelial growth factors (VEGF), the family of fibroblast growth factors (FGF), the family of platelet derived growth factors (PDGF), placenta growth factor (PlGF) and prostaglandins, etc. Suitable monocyte attractants include but are not limited to monocyte chemoattractant proteins (MCPs), granulocyte macrophage colony stimulating factor (GM-CSF, granulocyte colony stimulating factor (G-CSF) and tumor necrosis factor (TNF-α). Suitable vessel wall destabilizers include but are not limited to matrix metallo proteinases, (MMPs), elastase and collagenase. Gene therapy agent may be delivered by means of a plasmid, a viral vector, a liposomal delivery system an/or by encapsulation into a protein delivery system.

In order to most effectively stimulate the angiogenic/arteriogenic processes, the stimulants are preferably delivered over a sustained period, such as between 0 to 180 days, and more typically between 10 to 60 days. Such sustained delivery may be accomplished in various ways, such as by embedding, seeding, binding, or coating the graft vessel (either a natural, pre-fabricated or in situ formed graft vessel) with the stimulants or by the topical application or the injection of stimulants at the selected points of contact or close proximity.

With topical application methodologies, a delivery system may be provided upon proper positioning and placement of the graft vessel vis-à-vis the target vessel or tissue area, wherein one or more stimulants are applied topically to the selected points of contact or close proximity there between over a sustained period of time. In certain embodiments of the subject methods, the above-described device designed to apposition vessels and to maintain the position of vessels in close proximity may be configured to contain and slowly release one or more stimulants or other agents over the sustained period. For precise delivery to a selected point of contact or close proximity, the stimulants may be deposited at particular locations on the device adjacent to such selected point of contact or close proximity.

In other embodiments, topical delivery is accomplished by depositing biodegradable microbeads containing the stimulants at the selected points of contact or close proximity. The microbeads, which may be employed alone or in conjunction with the vessel appositioning device, are configured to slowly release the stimulants. The microbeads may be deposited by means of a syringe in a direct access approach to the surgical area at the time of positioning, placing, securing or maintaining the graft vessel. Alternatively, the microbeads may be deposited via a catheter delivered to the targeted deposit site, either endovascularly or extravascularly by a port, at the time of positioning, placing, securing or maintaining the graft vessel or after the surgical or interventional procedure has been completed.

Still other embodiments of the subject methods involve the delivery of stimulants, particularly stimulants in the form of gene therapy agents, by means of injecting them into the areas of contact or close proximity between vessel-and-vessel or vessel-and-tissue. The gene therapy agents provide sustained availability of stimulants in the areas adjacent to the selected points of contact or close proximity. As such, the cells resident in these adjacent areas are modified by the delivered genes and, as a result, slowly express and release the stimulants over a period of time The delivery of such gene therapy agents may be done concurrently with the positioning, placement, securement or substantial maintenance of the graft vessel, or at a later time, preferably by means of catheter based techniques.

Referring now to FIGS. 2A and 2B, there is illustrated various stages of another method of the present invention involving the direct, interventional establishment of a fluid communication between a graft vessel and a target vessel or tissue area immediately subsequent to or after a certain period of time, e.g., days, weeks or months, establishing a bonded tissue site between the vessels and/or the vessel and tissue area. As with the method described with respect to FIGS. 1A-D, the blood-supplying graft vessel 2 is moved or placed adjacent to or appositioned or juxtaposed against the target native vessel 4 or tissue area, such that at least one or more selected points of tissue or surface area of the graft vessel 2 are caused to physically contact or reside in close proximity to at least one or more corresponding selected points 8 of the exposed surface area of the native vessel 4 or of the targeted tissue area (see FIG. 1B). In certain embodiments, bio-adhesives are used to establish an immediate bond 14 between the external tissue surfaces of the two vessels at the one or more selected points. In other embodiments, tissue destabilizers may be applied to selected points 8 in order to degrade the epithelial layers of the respective vessels, thereby inducing immediate or subsequent encapsulation between the two vessels. In other embodiments, while it is intended that a fluid communication be established by the body's angiogenic and arteriogenic responses over a period of time, as discussed above, for some reason, such response does not occur or does not sufficiently occur to establish sufficient fluid communication to adequately perfuse the target vessel or tissue area. Such inadequate results may result where stimulants, such as those described with respect to FIG. 1C, are either not employed or are employed but do not cause the expected growth of capillaries and arterioles (such as capillaries and arterioles 16 provided in FIG. 1D). While inadequate connections are made between the vessels, the formation of one or more bonded tissue sites 14 via a naturally occurring encapsulation process may still occur, as illustrated in FIG. 2A.

In any of the just described situations, a physician may intervene, at the time of the initial procedure or at a time subsequent to the initial procedure, to better establish a supply of blood from graft vessel 2 to native vessel 4, such as by forming an opening, e.g., an arteriotomy, at the one or more bonded tissue sites 14 between the graft vessel 2 and the target vessel 4 so as to provide direct fluid communication, i.e., blood flow, from graft vessel 2 to target vessel 4 along the paths of arrows 20. Such opening is preferably established minimally invasively by means of a catheter 18 having a cutting element 22, e.g., a retractable scalpel, or a puncturing element, e.g., a retractable needle at a working end thereof. Such catheter 18 is delivered intervascularly through graft vessel 2 such that element 22 is operatively positioned at the bonded tissue site 22 and then activated to cut or pierce through bonded tissue site 22, upon which catheter 22 is removed from graft vessel 2. The delivery path of catheter 18 is most likely through one or more vessel passages, e.g., the aorta and/or another blood supplying vessel to which a graft vessel is connected or a graft vessel having a pre-existing or pre-established blood supply. The catheter may also be delivered through the target vessel itself, provided that crossing a blockage does not present an unacceptable risk to the patient. FIG. 2B, for example, illustrates delivery of an arteriotomy forming catheter 18 via graft vessel 2, having either a pre-existing or pre-established blood supply from the direction indicated by arrow 20, to the now bonded point of contact or close proximity 8. After properly positioning catheter 18 with the graft vessel, the cutting or puncturing element 22 is deployed to form an opening at the bonded site 8 such that blood 20 from graft vessel 2 is able to flow into target vessel 4.

The associated procedural steps of the above-described methods in the various above-described applications are preferably performed in the most minimally invasive manner possible. As such, a preferable approach for accessing and performing the requisite steps of the subject methods is by means of a percutaneous and/or port access approach involving the delivery of catheters and other minimally invasive instrumentation designed for particular functions, e.g., harvesting a graft vessel, positioning a graft vessel or repositioning a segment of a native artery, substantially maintaining the position of the graft vessel or said segment, applying or supplying stimulants to one or more targeted locations, establishing a blood supply to the graft and/or target vessel or tissue area, forming an arteriotomy at one or more bonding sites, viewing one or more such steps, etc. Many such functions may be performed by catheters and other endovascular and minimally invasive tools which are known to those skilled in the art.

Devices of the Present Invention

Certain devices of the present invention function to apposition or situate a blood-supplying vessel adjacent to another vessel, such as a blocked or stenotic vessel, or an area of tissue, such as a oxygen-deprived or necrotic tissue area, wherein one or more selected portions of the tissue surface of the blood-supplying vessel is caused to be or is placed in physical contact with or close proximity to one or more selected portions of the tissue surface of the other vessel or of the tissue area. Such contact or proximity between the tissue surfaces, either alone or in addition to an agent or stimulant provided at the contacted or proximated tissue surfaces, initiates the body's natural angiogenic and arteriogenic processes, over an expected period of time, to form a vascularized tissue bond between the outer tissue surfaces at the one or more selected points of contact or close proximity.

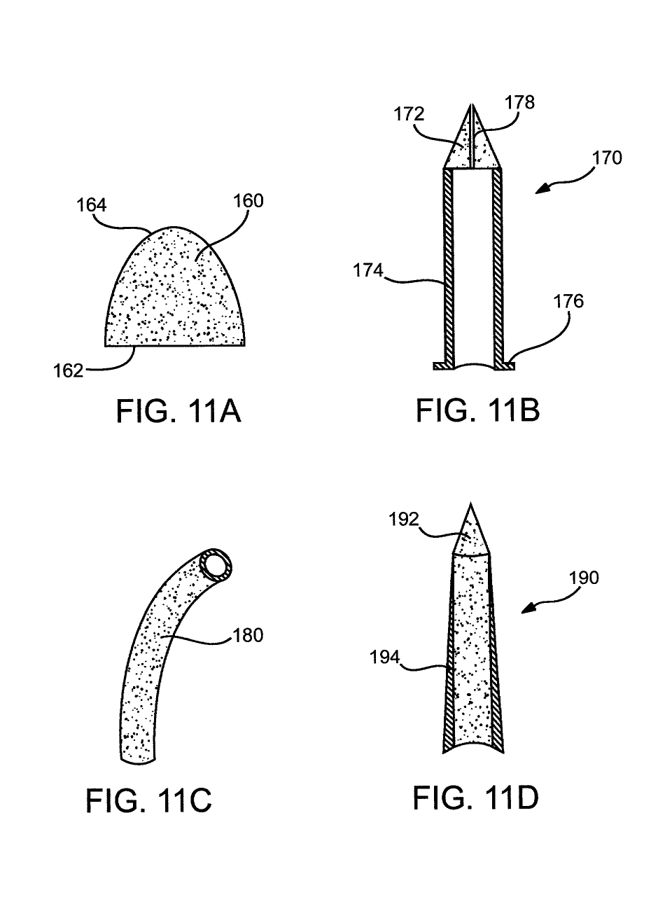

The tissue surfaces which may be appositioned by the devices of the present invention include but are not limited to the outer side walls of two vessels; the outer side wall of a graft vessel with the surface of tissue structure, such as the myocardium; and the transected end of one vessel, typically the graft vessel, with the side wall of another vessel, typically the target vessel. As such, certain of the devices of the present invention are configured to apposition or situate a side wall of vessels and other planar or curved tissue surfaces (see FIGS. 4-10), while others are configured to apposition a transected end of a side wall or planar or curved tissue surface (see FIGS. 11A-D).

FIGS. 4A and 4B illustrate an exemplary embodiment of a subject device of the present invention. Tissue contacting and appositioning device 60 is biocompatible and sized, shaped and configured to receive, accommodate or be in contact with at least two tissue structures, e.g., vessels, organs, tissue surfaces, to apposition or proximate the tissue structures at one or more points of contact or close proximity along their respective surface areas. More specifically, for example, tissue contacting device 60 is configured to receive surfaces areas of each of two vessels 68 and 70. As such, tissue contacting device 60 has a body or structure 62 having a first vessel or tissue contacting surface 64 and a second vessel or tissue contacting surface 66. Each tissue contacting surface 64, 66 is shaped to allow a tissue structure to be in flush contact with or in close proximity to body structure 62. Here, surfaces 64 and 66 are concave or curved and have a rectangular shape having length and width dimensions, respectively, to snugly accomodate and hold respective vessels 68 and 70 such that a least a portion of each of their surface areas are in close apposition to each other (see FIG. 4B). Surfaces 64 and 66 may each have any appropriate shape and dimensions to receive a corresponding tissue surface, vessel or organ. The two surfaces may have sizes and shapes different from each other. Additionally, device 60 may have more than two surfaces to receive and hold three or more tissue surfaces in contact with or in close proximity to each other. In most configurations, the device structures have a length in the range from about 3 to 10 mm and, if annular, have a diameter in the range from about 2 to 10 mm or, if planar, have a width in the range from about 5 to 15 mm.

Structure 62 further provides one or more openings 72 between structure surfaces 64 and 66 such that the surfaces of the tissue structures received on structure surfaces 64 and 66 are in physical contact with or in close proximity to each other at such one or more openings 72. The number of openings 72 within body 62 dictates the number of points of contact or close proximity between the appositioned tissue structures.

Additionally, body 62 may optionally have holding means, such as cover 74 associated with, e.g., hinged to, each structure surface to further secure and substantially maintain a tissue structure flush against the structure surface and thus substantially maintain the tissue structure in contact or close proximity with the apposing tissue structure(s). Cover 74 may have a locking mechanism (not shown), e.g., a clip or snap fit mechanism, to lock it to structure 62 when in a closed position. Cover 74 has an internal surface 76 suitably sized and shaped to contact the tissue structure it encloses within device 60. Cover 74 may be made of a flexible material so that it may conform to the surface of the tissue structure that it contacts. Here, device 60 has a single cover 74 associated with first surface 64 and having a surface 76 having a curved, rectangular shape, such that when cover 74 is in a closed position, as illustrated in FIG. 4B, it and surface 64 define a lumen through which vessel 70 is received. On the other hand, because graft vessel 70 is not fixed, and thus subject to dislodgement from device 60 by the beating of the heart, cover 74 provides further securement of graft vessel 70 which will prevent graft vessel 70 from moving away from native vessel 68. In this application, a second cover associated with second surface 66 is not necessary or useful, as vessel 68, a native coronary artery, is embedded within the epicardial surface 80 of the heart. However, if the particular application involves appositioning two wholly exposed or free vessels, the use of a second cover may be appropriate. Any suitable number of covers may be employed by the tissue contacting device to secure any number of tissue structures against each other or in close proximity to each other. Alternatively or additionally, a bio-adhesive may be applied to the tissue contacting surfaces of device 60. For example, a bio-adhesive may be applied to second surface 66 to secure it over the intended target section of tissue and to prevent it from moving as a result of the beating of the heart.

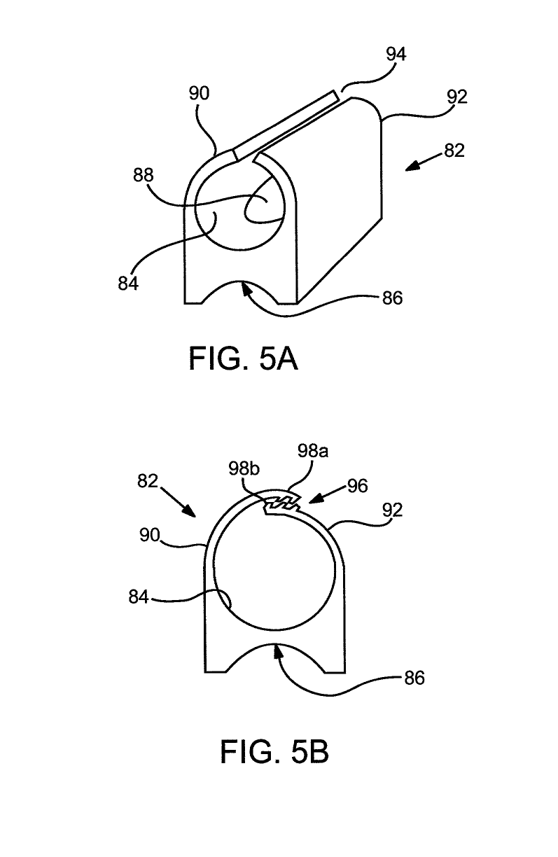

FIGS. 5A and 5B illustrate another embodiment of a tissue appositioning device 82 of the present invention having a configuration and dimensions similar to that of device 60 of FIGS. 4A and 4B. Device 82 is provided with a first vessel or tissue contacting surface 84, a second vessel or tissue contacting surface 86 and an opening 88 there between such that the surfaces of the tissue structures received on surfaces 84 and 86 are in physical contact with or in close proximity to each other at opening 88. While second surface 86 is similar to second surface 66 of device 60 of FIGS. 4A and 4B, first surface 84 has sides walls 90 and 92 which extend upward and curve inward towards each other so as to form an annular lumen configuration, yet having a gap 94 between the distal ends of side walls 90 and 92. Such a configuration may be described as a holding means for holding a vessel or other tissue structure. Device 82 may be made of a material such that side walls 90 and 92 are sufficiently flexible to accommodate vessels of varying diameters and to be spread apart to allow easy placement of a vessel within the lumen defined by side walls 90 and 92.

Device 82 may further include a locking mechanism 96 for joining side walls 90 and 92 together, as illustrated in the cross-sectional view of device 82 FIG. 5B, in order to better secure a vessel held within first surface 84 to device 82 and to avoid dislodgement caused by the beating of the heart, for example. FIG. 5B illustrates an exemplary locking mechanism 96 as sets of teeth 98 provided at the respective distal ends of side walls 90 and 92. More specifically, side wall 90 has two parallel rows or sets of angled teeth 98a provided on the inside surface of side wall 90. Side wall 92 has three parallel rows or sets of angled teeth 98b provided on the outside surface of side wall 92. When side walls 90 and 92 are compressed together, angled teeth 98a and angled teeth 98b interlock with each other, thereby locking together the side walls 90 and 92 to form a closed lumen. Angled teeth 98a and 98b may extend continuously along the entire length of the associated side wall or may be provided in any number of segmented pairs aligned along the length of the associated side wall. Any number of sets or rows of teeth may be used as long as there is at least one tooth associated with each side wall.

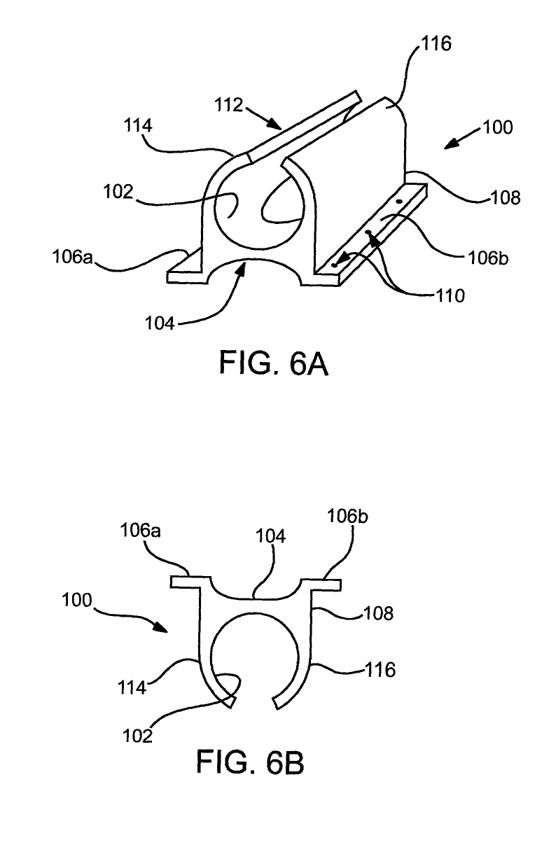

FIGS. 6A and 6B illustrate yet another embodiment of an appositioning device 100 having first and second tissue contacting surfaces 102 and 104 which respectively have the same configuration as the corresponding surfaces of device 82 of FIGS. 5A and 5B wherein first surface 102 is defined by holding means 112. Similar to the device of FIGS. 5A and 5B, holding means 112 includes side walls 114 and 116 which are flexible or have elastic properties to allow for easy placement of a vessel therein and to securely maintain the vessel thereafter. Device 100 has extensions or feet 106a and 106b extending from body 108 along the length of device 100. Each extension may be provided with one or more holes or bores 110 through which a suture may be threaded to secure device 100 to the surface of a tissue structure. Such a configuration is particularly useful when device 100 is located in a position where it is subject to gravity or significant movement within the body and, thus, requires a very secure means of maintaining its position.

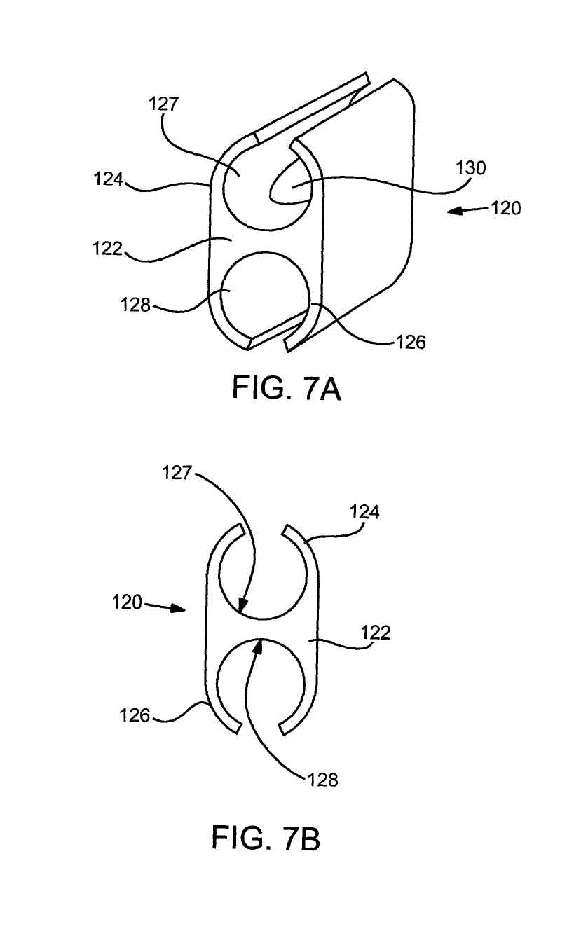

FIGS. 7A and 7B illustrate another embodiment of an appositioning device 120 having a body structure or portion 122 and first and second vessel holding means 124 and 126 on opposing sides of structure 122, each defining a vessel contacting or appositioning surface 127 and 128, respectively. An opening 130 is provided within a central portion of structure 122 such that vessels positioned within holding means 124 and 126 are in contact or in close proximity to each other at opening 130. Such a configuration is suitable where two free vessels, rather than a single vessel and a tissue surface area, are to be appositioned together, such as in peripheral vascular applications, e.g., where a free graft vessel is appositioned to a diseased peripheral vessel, e.g., radial artery to femoral or popliteal artery, saphenous vein to either carotid, femoral or popliteal artery. With applications which employ a device 120, it is not as necessary to fix or adhere the device to a tissue surface; however, if such is preferred, an adhesive may be applied to one or more outer surface areas of device 120. The dimensions (e.g., width, length and diameters) and physical properties (e.g., flexibility) of device 120 are equivalent to the apposition devices previously discussed. Alternatively, device 120 may have a tubular or hollow cylindrical structure wherein the outer wall of the device is contiguous and a lumen is defined therein (i.e., no central portion is present). The lumen has a diameter sufficient to accommodate said graft tissue structure and said at least one target tissue structure in contact or close proximity within said lumen. For example, the two tissue structures may be placed side-by-side in a parallel relationship.

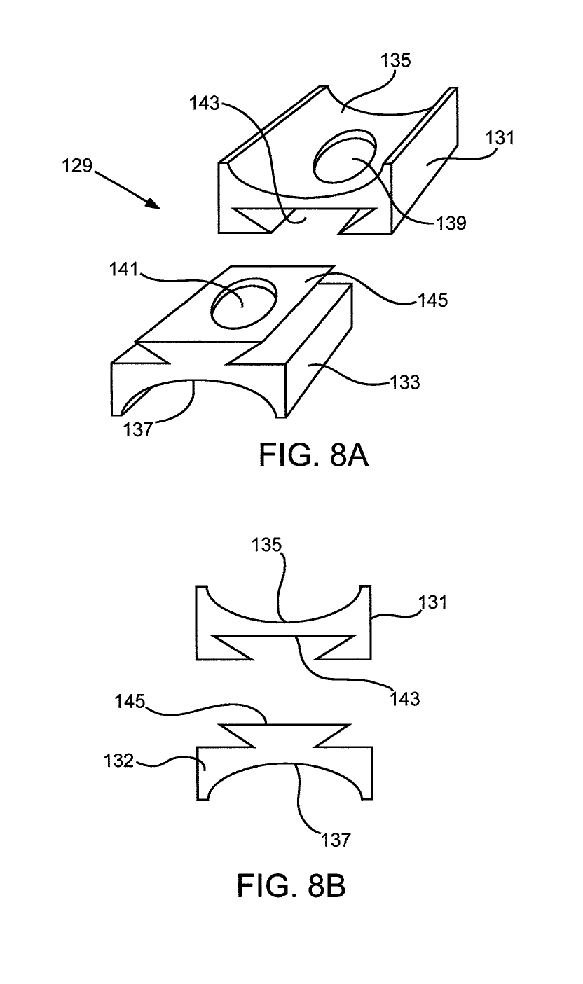

FIGS. 8A and 8B illustrate another apposition device 129 havng a two-piece configuration. A first piece or member 131 and a second piece or member 133 each have a tissue contacting surface 135 and 137, respectively, and on opposite sides of the tissue contacting surfaces are interfacing structures 143 and 145, respectively. Interfacing structures 143 and 145 each have a configuration to matingly engage with the other. For example, they may have opposing tongue and groove configurations, such as tongue 145 and groove 143. Through the structure of each piece 131 and 133 is an opening 139 and 141, respectively, where the two openings have the same dimensions. When members 131 and 133 are operatively engaged together, openings 139 and 141 are aligned to allow the appositioned vessels to contact or be in close proximity with each other.

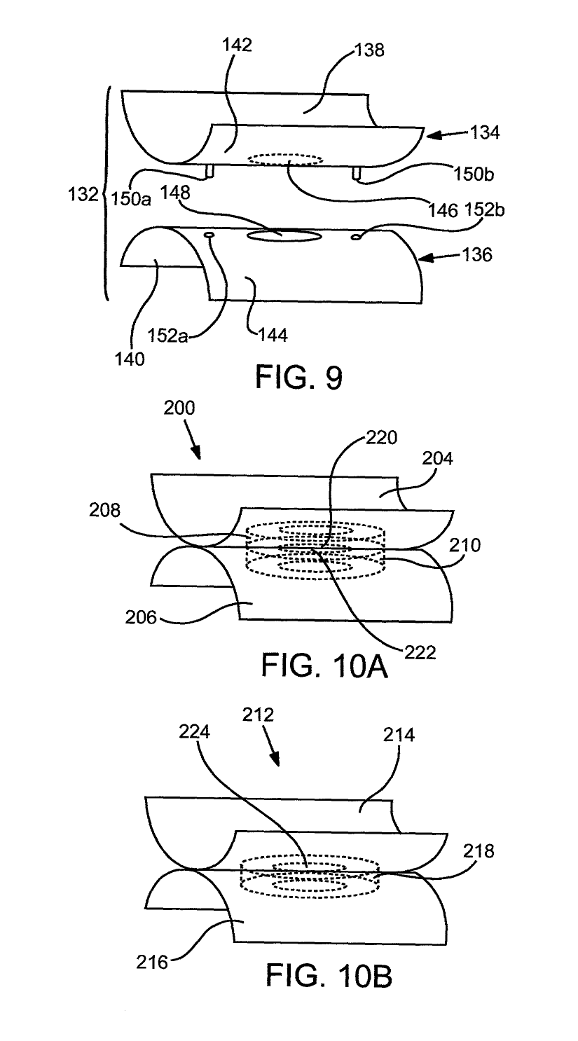

FIG. 9 illustrates another two-piece vessel apposition device 132. A first piece 134 and a second 136 have very similar configurations, although they may differ from each other as necessary to accommodate a particular tissue structure. First and second pieces 134 and 136 each have a tissue contacting surface 138 and 140, respectively, and an outer surface 142 and 144, respectively. Each piece has an opening 146 and 148, respectively, such that when the two pieces are operatively coupled together, openings 146 and 148 substantially align with each other. Each piece has at least one fastener portion for coupling or mating with the fastener portion of the other piece. For example, first piece 134 has two fastener portions 150a and 150b having a pin, dowel, tab, peg or other like configuration, while second piece 136 has corresponding fastener portions 152a and 152b having a hole, bore, receptacle, recess or other like configuration for receiving fastener portions 150a and 150b, respectively. Fastener portions 150a and 150b, respectively, are aligned on the outer surface 142 to engage with fastener portions 152a and 152b, respectively, such that their frictional or snap-fit engagements sufficiently secure pieces 134 and 136 together, and thereby align openings 146 and 148 with each other such that a tissue structure operatively held or positioned within first piece 134 is in contact with or in close proximity to a target surface area of a second tissue structure held within second piece 136. Such a device configuration is also suitable for applications that do not require the device to be fixed or adhered to adjacent tissue structures.

FIGS. 10A and 10B illustrate other embodiments of appositioning devices of the present invention. Device 200 of FIG. 10A has a two-piece configuration which includes a first tissue holder 204 and a second tissue holder 206, having a construct similar to that of device 132 of FIG. 9; however, here, the tissue holders, and thus the vessels to be appositioned, are held together by a magnetic force created by a first magnetic means 208 and a second magnetic means 210, respectively, associated with first tissue holder 204 and second tissue holder 206, respectively. The embodiment 212 of FIG. 10B is similar to that of FIG. 10A, having the same two-piece vessel holder configuration; however, only one tissue holder 216 is provided with magnetic means 218 while the other vessel holder 214 is made of a material, e.g., metal, which is attractable to a magnetic force. In either embodiment, the magnetic means is attached to the respective vessel holder by either mechanical or adhesive means or is embedded within the holder where the vessel holder is made of a non-magnetic material, or by magnetic attraction where one or both vessel holders are made of a metal or other magnetically attractable material. The magnetic force between the two magnetic means 208 and 210 of FIG. 10A or between the single magnetic means 218 and the opposing vessel holder 214 of FIG. 10B is sufficient to maintain the respective pairs of tissue holders substantially motionless with respect to each other (so as not to interrupt the angiogenic and arteriogenic processes). Suitable magnetic means as well as electrostatic means and mechanical means for use with the present invention are disclosed in U.S. Pat. No. 6,352,543 B1, which is herein incorporated by reference.

In both embodiments, the magnetic means has a shape and configuration which does not in anyway obstruct the respective openings 220 and 222 of FIG. 10A and opening 224 of FIG. 10B within the respective vessel holder means. For example, the magnetic means may have an annular or ring shape having an opening therein, preferably having an inner diameter which substantially corresponds with the diameters of vessel holder openings. Further, the magnetic means are very thin so as to allow the subject tissue structures to be substantially flush with the surfaces of the respective tissue holders. The magnetic means may be placed within the inner or concave side of a tissue holder or on the outer or convex side of a tissue holder and/or may be flush with one or both sides.

As mentioned above, the tissue appositioning devices of the present invention may have any suitable configuration. For example, the device may include a series of support bands or strips wherein two appositioned tissue structures contact each other at open areas between the bands or are maintained in a position of close proximity where these open areas allow for the formation of a connection between the tissue structures. The devices may alternatively have a grid pattern defining open cells wherein appositioned tissue structures contact each other at multiple points of contact. Where the tissue structures to be bonded are both loose or unsecured, the devices may, instead of having tissue contacting surfaces on opposite sides of an opening, define an open space having opposing walls wherein two or more tissue structures can be held directly against each other between the opposing walls. Yet another configuration of a device may provide one or more straps or clips for substantially maintaining two tissue structures together in a manner sufficient to allow the angiogenesis and arteriogenesis processes to occur between them. As a further alternative, vessels or tissue structures may be sutured to the surface of the devices such that their position on the respective surfaces is maintained. Suturing may be used in addition to or instead of the above methods of positional fixation.

The size and shape of the tissue contacting surface areas of the subject devices will depend on the sizes and shapes of the corresponding tissue areas to be bonded. For example, an appositioning device may have dimensions to snugly accommodate a vessel, such as a saphenous vein graft or a coronary artery, having inner diameter dimensions suitable for accommodating vessels in the range from about 1.5 to 6 mm, and more typically in the range from about 2 to 4 mm, but may larger or smaller depending on the vessel size or the surface area of the tissue surface to be contacted. The shape of the tissue contacting surface areas may be annular or lumenal, having any appropriate circumference length and radius of curvature, or may be planar or flat to be flush with a corresponding planar tissue surface area, for example, the surface of the myocardium such as when the application involves a target coronary artery which is embedded within the myocardium. The size of the one or more apposition openings will also depend, for example, on the size of the tissue structures or surface areas to appositioned. Also, if the intent is or it is later necessary to create an arteriotomy at a bonded tissue site, the length of the arteriotomy should be considered. The length of arteriotomies for coronary artery bypass procedures, for example, are in the range from about 1 to 15 mm, and more typically from about 4 to 10 mm.

FIGS. 11 A-D illustrate other embodiments of appositioning devices of the present invention wherein the device structures are comprised of a substrate material. The substrate material has a porous structure, preferably having a porosity from about 50% to 90%, and preferably a mean pore size in the range from abut 5 um to about 250 um which serves as a scaffold for tissue in-growth to anchor the substrate device to a graft tissue structure and/or to a target tissue structure and thus promote angiogenic and arteriogenic growth between the tissue structures. The substrate device may be made of either a biodegradable or nonbiodegradable material but is preferably made of a biodegradable material, such as a hydrogel material or a biological or synthetic polymer. With a biodegradable configuration, angiogenic and arteriogenic processes take over as substrate device biodegrades to form a more natural and permanent connection between the tissue structures. Such porous structure may contain of one or more agents for stimulating the formation of a vascularized connection between a graft tissue structure and target tissue structure. Additionally, the substrate material may be seeded as mentioned above with stimulants or tissue destablizers to promote this growth.

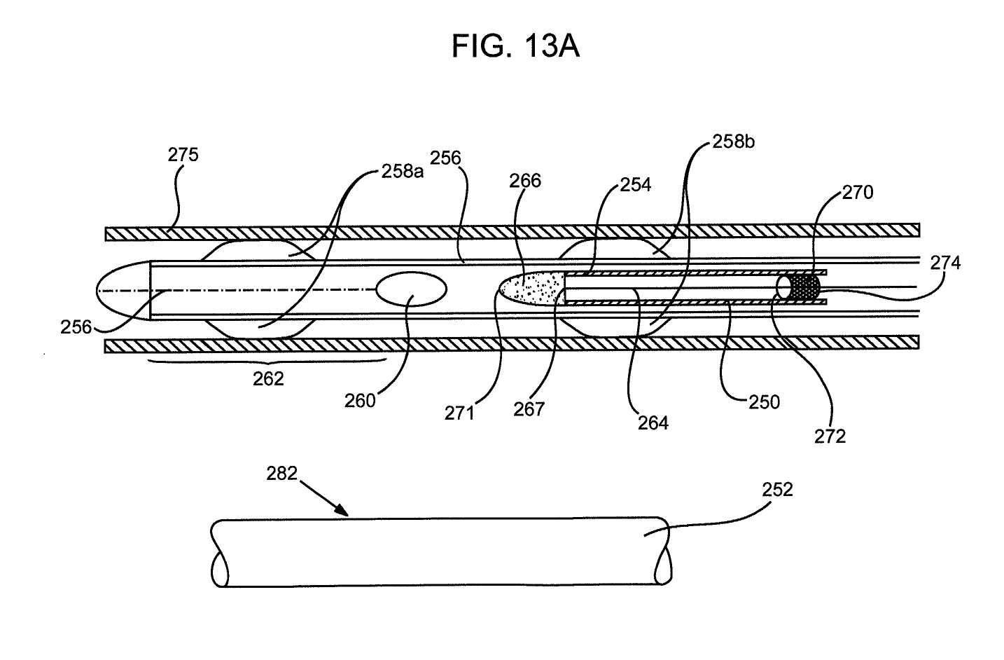

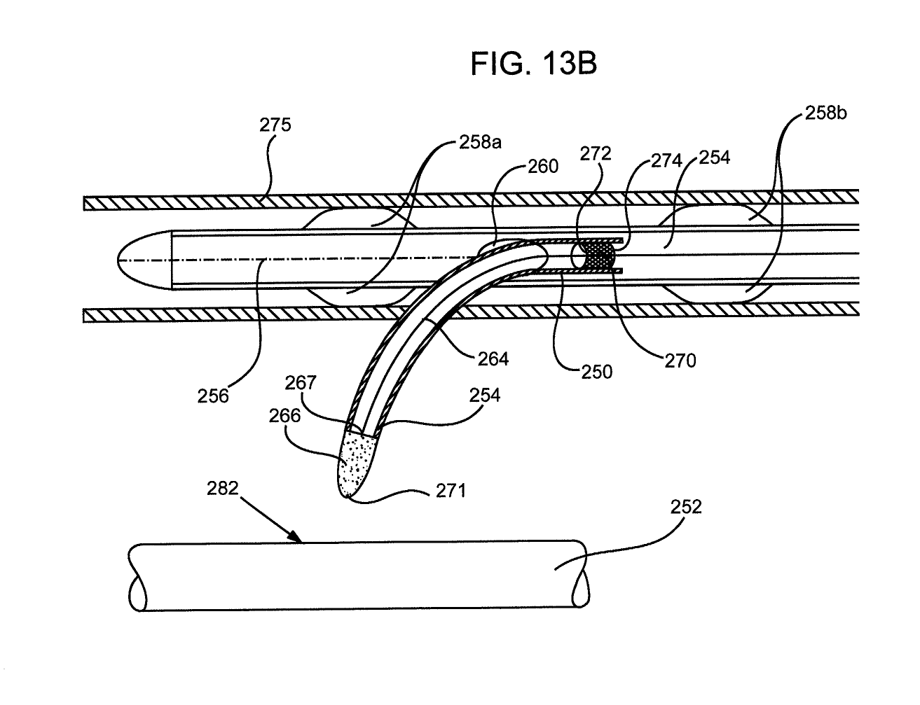

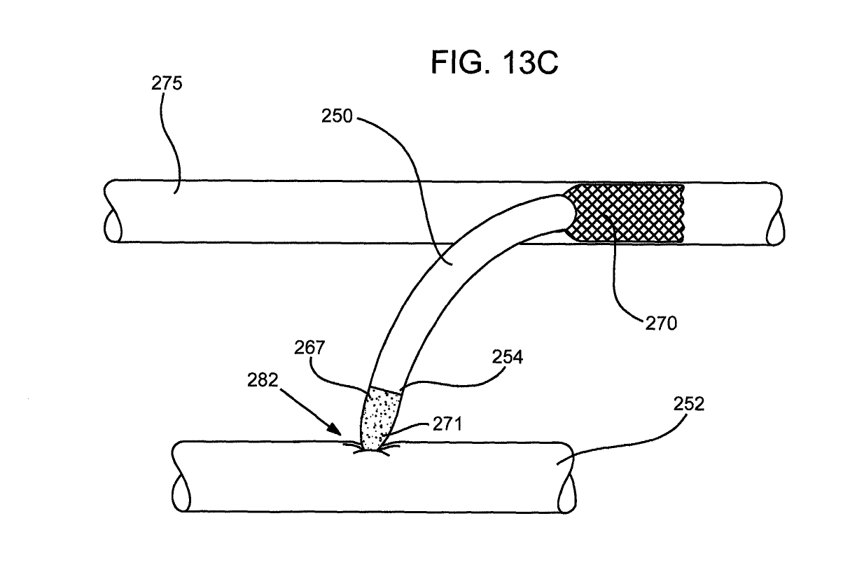

The substrate material may have any suitable configuration, size and shape. For example, appositioning device 160 has a bullet shape where a proximal end 162 has a planar configuration to be affixed, such as by a bio-adhesive, to a dissected end of a vessel, such as a graft vessel as illustrated in FIGS. 13A-C. Distal end 164 has a curved configuration to make contact with a tissue structure or vessel, such as a target vessel or tissue structure. A bio-adhesive may also be used at distal end 164 to establish an initial contact with the tissue structure. As mentioned above, the porosity of device 160 facilitates in-growth of tissue with the likely result being that the two appositioned vessels and/or tissue structures eventually form a tissue bond. Alternatively, the proximal end 162 is hollow such that the inside of device 160 receives the end of a vessel such as a graft vessel.

The substrate device may have a pointed or nose cone configuration, such as illustrated in FIGS. 11B and 11D, for navigating through a delivery catheter. The device may have a sharpened tip to penetrate through tissue, such as a vessel wall, as well as to slightly penetrate or stick to the surface of a target tissue structure, thus facilitating the initial apposition or engagement of the distal end of a graft vessel to another tissue structure, such as illustrated. In FIG. 11B, apposition device 170 includes a substrate portion or structure 172 having a pointed nose cone configuration. Optionally, substrate portion 172 may have a narrow lumen 178 for accommodating a guide wire for delivery purposes. Attached at a proximal end is a tubular structure, sheath or conduit 174 which may be made of the same or different material as structure 172. Tubular structure 174 has an internal diameter dimension to accommodate or contain a distal end or the entire length of a graft vessel, wherein device 170 is implanted into a bed of tissue or through a thick tissue wall, such as the wall of the left ventricle. Tubular structure 174 may be configured to be load-bearing or self-expanding so as to securely retain itself once implanted or embedded within the tissue structure. Such sheaths or conduits suitable for use with the present invention are disclosed in PCT International Publication Numbers WO 00/21436, WO 00/21463 A1 and WO 00/41632 A1 and U.S. patent application Publication Number US 2001/0025643 A1, which are herein incorporated by reference. Tubular structure 174 may further include an anchor or stop mechanism, such as radially extending lip 176, to limit penetration of device 170.

The apposition device 180 of FIG. 11C comprises the substrate material in a tubular configuration. Optionally, tubular structure 180 may have one or more curves or angles to facilitate delivery of or appositioning of tissue structures. In FIG. 11D, apposition device 190 has a tapered tubular structure 194 which tapering extends to a pointed distal tips structure 192 to further facilitate delivery if penetration through tissue structures is necessary.

Any of the above described apposition devices may be formed of any suitable permanently implantable, non-biogradable materials or of bioresorbable or biodegradable materials. Non-biodegradable materials useful in fabricating the devices of the present invention include but are not limited to plastics, silicones and metals. Suitable plastics include but are not limited to polytetrafluoroethylene, polyethylene terephthalate, polyvinyl alcohols, or a plastic from the family of polyurethanes, polyesters and polyethylene. Suitable metals include but are not limited to titanium, nitinol, stainless steel, or the like.

Suitable biodegradable materials useful for the devices of the present invention include but are not limited to biodegradable polymers and biodegradable or bioactive glass. Suitable biodegradable polymeric materials include, but are not limited to, polyglycolide (PGA), copolymers of glycolide, glycolide/L-lactide copolymers (PGA/PLLA), lactide/trimethylene carbonate copolymers (PLA/TMC), glycolide/trimethylene carbonate copolymers (PGA/TMC), polylactides (PLA), stereo-copolymers of PLA, poly-L-lactide (PLLA), poly-DL-lactide (PDLLA), L-lactide/DL-lactide copolymers, copolymers of PLA, lactide/tetramethylglycolide copolymers, lactide/α-valerolactone copolymers, lactide/ε-caprolactone copolymers, hyaluronic acid and its derivatives, polydepsipeptides, PLA/polyethylene oxide copolymers, unsymmetrical 3,6-substituted poly-1,4-dioxane-2,5-diones, poly-β-hydroxybutyrate (PHBA), PHBA/β-hydroxyvalerate copolymers (PHBA/HVA), poly-p-dioxanone (PDS), poly-α-valerlactone, poly-ε-caprolactone, methacrylate-N-vinyl-pyrrolidone copolymers, polyesteramides, polyesters of oxalic acid, polydihydropyranes, polyalkyl-2-cyanoacrylates, polyurethanes, polyvinylalcohol, polypeptides, poly-B-malic acid (PMLA), poly-B-alcanoic acids, polybutylene oxalate, polyethylene adipate, polyethylene carbonate, polybutylene carbonate, tyrosine based polycarbonates, chitin derivates such as chitosan and other polyesters containing silyl ethers, acetals, or ketals, alginates, and blends or other combinations of the aforementioned polymers. In addition to the aforementioned aliphatic link polymers, other aliphatic polyesters may also be appropriate for producing aromatic/aliphatic polyester copolymers. These include aliphatic polyesters selected from the group of oxalates, malonates, succinates, glutarates, adipates, pimelates, suberates, azelates, sebacates, nonanedioates, glycolates, and mixtures thereof. In addition, biodegradable materials may comprise proteins such as fibrin, collagen, elastin or the like. The synthesis and formulation of biodegradable implant compositions for selected mechanical properties are well known to those skilled in the art, and the aforementioned materials may be utilized to prepare compositions suitable for use with the invention.

Additionally, the devices of the present invention may have at least one radio-opaque marker incorporated into the structure of the device for purposes of identifying the device under fluoroscopic observation.

As mentioned above, the devices of the present invention may further function to provide or deliver agents, such as stimulants or tissue destablizers, as described above, to one or more of the tissue structures being contacted or appositioned. For example, tissue appositioning surfaces of the devices, especially in the area closest to the apposition openings, may be coated or embedded with such agents for either immediate release or release over a period of time.

Systems of the Present Invention

The systems of the present invention may include instrumentation and devices for surgically and/or percutaneously (by a catheter-based approach) accessing, presenting and selectively placing one or more vessels in close proximity to or in physical contact with each other. Certain such systems include instrumentation and devices for providing a graft vessel, e.g., a pedicled artery in contact with or in close proximity to the heart, an in situ graft, or a section of vessel harvested from elsewhere in the patient or from a donor or pre-fabricated or fabricated in situ, in contact or close proximity to a stenotic or occluded vessel at a selected location distal to the stenosis or occlusion. The systems may further include devices for maintaining such contact or close proximity until the body's angiogenic and arteriogenic processes forms one or more vascularized tissue bonds between the vessels, and establishing fluid communication between the vessels at the bonded sites.

Additionally, the systems of the present invention may include one or more subject devices facilitate positioning or appositioning one vessel or tissue structure adjacent another vessel or tissue structure. Such systems include “self-guiding” capabilities. For example, magnetic material may be incorporated within the subject appostioning devices (such as those described above with respect to FIGS. 10A and 10B), within the delivery devices used for positioning one vessel or tissue structure adjacent to another vessel or tissue structure and/or within the instrumentation used to establish fluid communication between the naturally connected vessels.

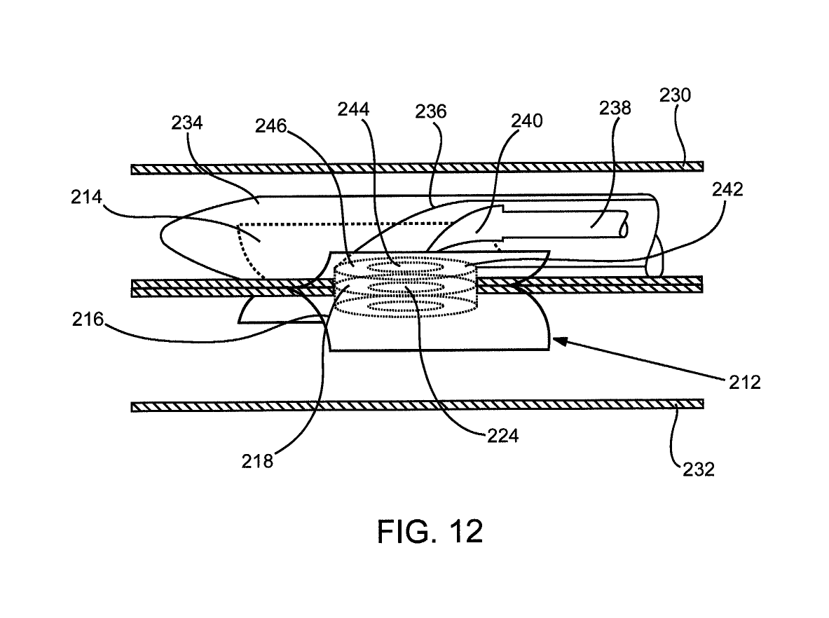

Referring now to FIG. 12, there is illustrated a system of the present invention for creating a fluid communication opening or arteriotomy between two tissue structures, e.g., vessels 230 and 232. Vessels 230 and 232 have been previously appositioned or placed in contact with or at one or more points of close proximity to each other by means of an appositioning device 212, discussed in detail above with respect to FIG. 10B. Specifically, vessel 230 is held within vessel holder 214 and vessel 232 is held within vessel holder 216. The vessel holders are held together by magnet means 218 which resides within vessel holder 216. Such previous placement or appositioning may have been performed immediately prior and within the same operation as the steps for forming a fluid communication between the vessels, or may have been preformed substantially earlier, such as days, weeks or months earlier. The latter situation arise, for example, when a natural tissue bond forms, as described above with respect to FIGS. 2A and 2B, between a blood-supplying graft tissue structure 230 and a target tissue structure 232 but is insufficiently vascularized to provide a sufficient blood supply to target tissue structure 232 and the surrounding tissue area.

The system of FIG. 12 includes a steerable delivery catheter 234 having dimensions so as to be deliverable within vessel 230. Catheter 234 has a lumen 236 therein through which a tissue incising tool 238 is translatable. Incising tool 238 has an incising, cutting, piercing or puncturing element 240 at its distal end. Lumen 236 terminates distally at a lumen port or opening 242 at which there is affixed a docking means or port 244 which may include magnetic means 246, electrostatic means or mechanical means of the type disclosed in U.S. Pat. No. 6,352,543 B1. Magnetic means 246 has a configuration, e.g., annular configuration which may be continuous or non-continuous i.e., made of discrete segments, which allows tissue incising element 240 to be delivered through lumen port 244 and to align with magnetic means 218 of vessel holding member 216. The magnetic force between magnetic means 218 and 246 when in close proximity, cause magnetic means 246, and thus catheter 234, to become docked to appositioning device 212 at opening 224. Incising tool 238 is then manipulated to cause incising element 240 to cut through the bonded tissue site and thereby create a fluid passage opening between vessels 230 and 232 such that blood may flow from vessel 230 to within vessel 232.

FIGS. 13A-C illustrate a system of the present invention for endovascularly or intravascularly delivering a free graft tissue structure, here illustrated as free graft vessel 250 to a target tissue structure or area, here illustrated as a vessel 252, and appositioning the distal end 254 of graft vessel 250 at a selected point of contact or close proximity 282 on target vessel 252. The system includes a steerable delivery catheter 256 having dual occlusion members, such as expandable balloons 258a and 258b, and an exit port 260 there between. The portion 262 of delivery catheter 256 distal to exit port 260 includes a break-away or peel-away sheath configuration. Catheter 256 has dimensions so as to be deliverable through the lumen of a conduit, such as the internal mammary artery, the gastroepiploic artery, the aorta or the like.

The system further includes a steerable graft vessel guide wire or catheter 264 or the like having an appositioning or apposition device in the form of an implantable substrate 266 releasably attached at a distal end of guide wire 264. Substrate 266 is configured on a proximal side or end 267 to be engaged or adhered to distal end 254 of graft vessel 250. Substrate device 266 is configured at distal end 271 to engage with or adhere to a tissue structure 252. To this end, substrate device 266 may have a pointed or nose cone configuration for navigating through delivery catheter 256, and may have a sharpened tip to penetrate through the wall of aorta 275 as well as to slightly penetrate or stick to the surface of the target tissue structure 252, thus facilitating the initial apposition or engagement of the distal end 254 of graft vessel 250 to another tissue structure 252. Substrate device 266 may further be provided with a bio-adhesive to facilitating this initial engagement.

The system further includes a self-expanding anchoring means, such as stent 270, which is inserted within the proximal end of free graft vessel 250. Stent 270 is compressible to a low-profile state for delivery through delivery catheter 256 over guide wire 264 and is expandable upon deployment from this compressed state to provide a “lap-joint” connection between the delivery/supply conduit 275 and graft vessel 250 (see FIG. 13C).

The system of FIGS. 13A-C is employed as follows. Free graft vessel 250, which may be either autologous, a donor vessel or bioengineered, is provided and operatively attached at its distal end 254 to the proximal end 267 of substrate 254 and at its proximal end to stent 270, wherein graft vessel 250 and stent 270 are positioned about and deliverable over guide wire 264. The assembly is then inserted into the proximal end of delivery catheter 256 which has been previously or is simultaneously delivered to a target location within a blood supplying vessel 275, such as the aorta or IMA. At the target location, as shown in FIG. 13A, occlusion balloons 258a and 258b of delivery catheter 256 are expanded or inflated so as to temporarily occlude blood flow through the supply vessel and thereby provide a blood-free area at the target location.

Next, as shown in FIG. 13B, an opening is formed within the wall of blood supply vessel 275 either by means of substrate 266, if configured with a sufficiently sharp tip 271, which is maneuvered through exit port 260 of catheter 256 to contact and cut through the wall of vessel 275. Alternatively, prior to the delivery of the graft vessel assembly, another incising or piercing instrument (not shown), such as a guide wire having a needle or blade configuration at its distal end, is delivered through delivery catheter 256 and through exit port 260 to pierce or cut through the wall of vessel 275 (not shown).

After creating such vessel wall opening, the cutting instrument is retracted from catheter 256. Preferably, at this point, catheter 256 is adjusted, if necessary, such that exit port 260 is substantially aligned with and adjacent to the vessel opening just formed. Next, substrate 266, and the attached graft vessel 250 and stent 270, are translated in a forward direction and delivered through exit port 260 and the vessel opening to a target point of contact or close proximity 282 on target tissue structure 252 (see FIG. 13B). As distal end 272 of stent 270 exits vessel opening 280 and proximal end 274 of stent 270 exits exit port 260, it is released from its compressed condition and allowed to deploy, thereby operatively connecting the proximal end of graft vessel 250 to the side wall of supply vessel 275, thereby interconnecting the two vessels. As illustrated in FIG. 13C, substrate 266 is then further steered and moved, as necessary, by manipulation of guide wire 264 to cause it to engage or adhere, as discussed above, to the outer wall or surface area tissue structure 252 at point 282. Upon proper engagement or adherence between substrate 266 and tissue structure 252, guide wire 264 is operatively released from substrate 266 and removed from graft vessel 250 back into the lumen of catheter 256. Occlusion balloons 258a and 258b are then deflated, and catheter 256 is withdrawn from blood supply vessel 275. Upon withdrawal of catheter 256, sheath 262 is caused to separate and peel apart.

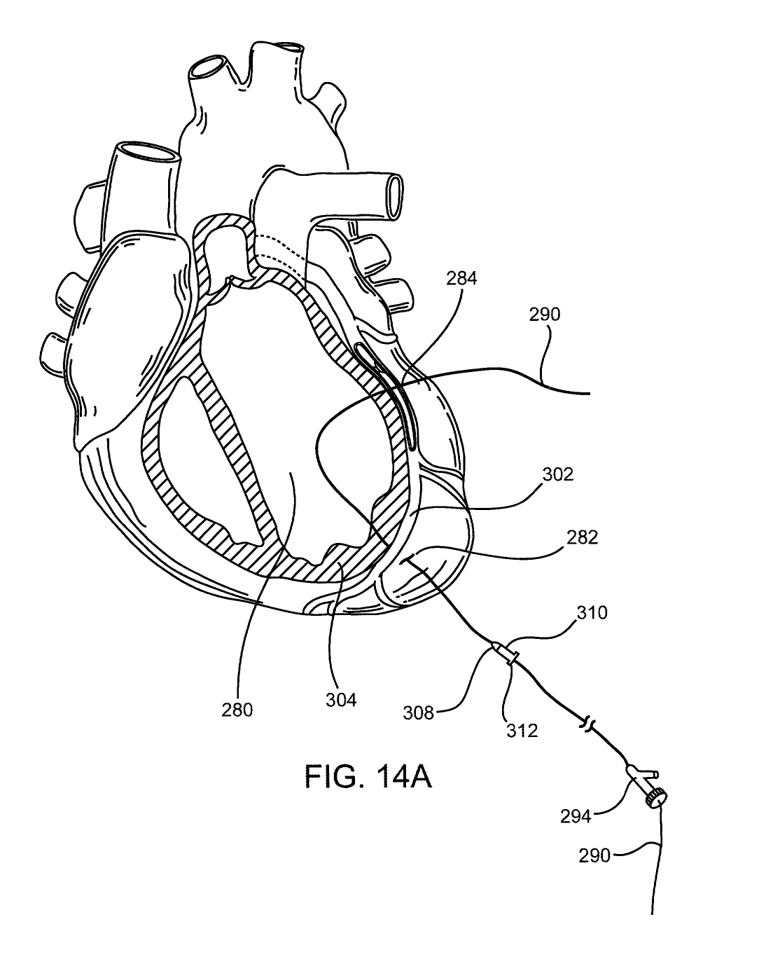

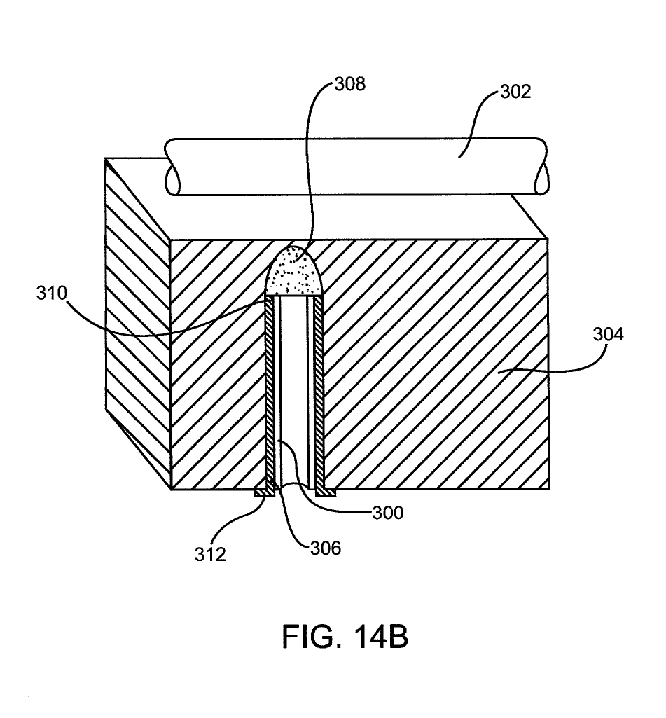

FIGS. 14A and 14B illustrate a method of the present invention for delivering an appositioning device, which is similar to device 170 of FIG. 11B, having an attached free graft vessel 300 (shown in FIG. 14B), to a target tissue structure or area, such as a target coronary artery 302. The Seldinger technique may first be used to establish access to target vessel 302 wherein a needle mechanism or guide wire 290 is introduced into the chest cavity of the patient through an introducer sheath 294 and is used to puncture into the left ventricle 280 at a first location or entry site 281. The guide wire 290 is then advanced to and penetrated through a second location or implant site 284 of the left ventricle wall. Next, the apposition device having graft vessel 300 coaxially loaded within tubular structure 310 is delivered over guide wire 290 through left ventricle entry site 284 and over the guide wire 290 up back to the first entry site 281 such that substrate tip 308 is operatively appositioned at a selected point of contact with or close proximity to target vessel 302. While a direct delivery approach has been illustrated, apposition device 310 may also be delivered intravasclarly, for example, over a guide wire which has been delivered via a cut down incision within the femoral artery in the groin area, over the aortic arch and crossing the aortic valve into the left ventricle. The ventricle wall is then penetrated at a selected location wherein the device may be implanted.

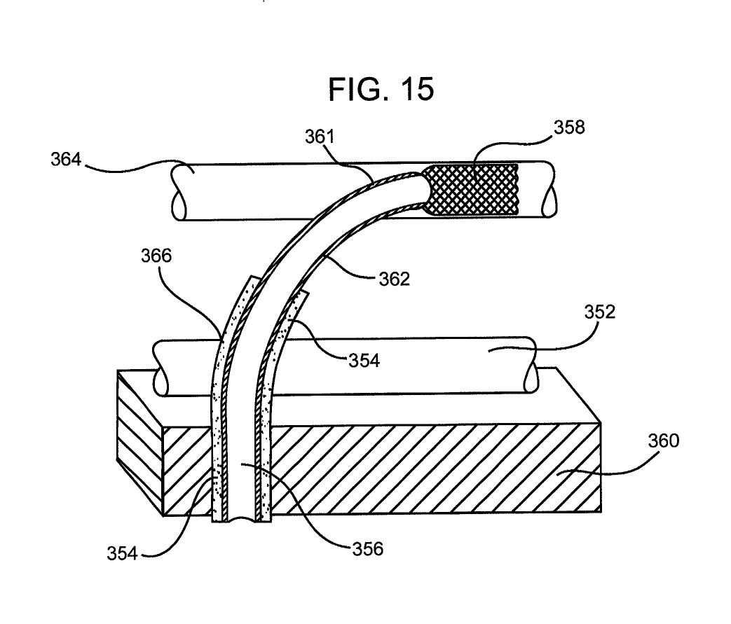

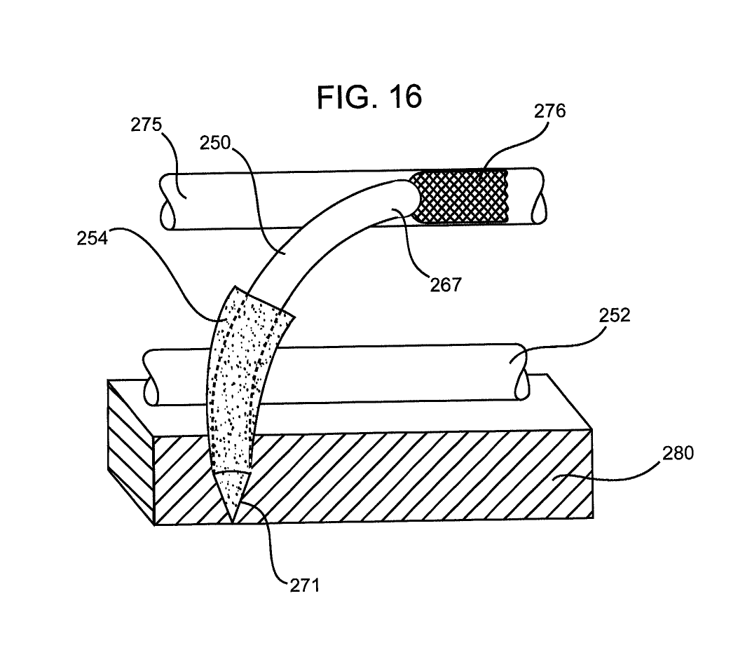

FIGS. 15 and 16 illustrate the device embodiments of FIGS. 11C and 11D, respectively, having been delivered with the system of FIGS. 13A-C. The procedures for implanting the devices of FIGS. 15 and 16 may be performed by either of the approaches described with respect to FIGS. 13A-C or FIGS. 14A and 14B.

In FIG. 15, a porous apposition device 366, having a distal end 356 of free graft vessel 362 loaded therein, has been implanted within the wall 360 of the left ventricle at a location near a target vessel 352, such as a coronary artery located at the surface of the left ventricular wall. The proximal end 366 of device 366 extends outside the ventricular wall 360 and overlies target vessel 352, at a selected point of contact or close proximity. The proximal end 361 of graft vessel 362 has been affixed to a blood-supplying vessel 364 by self-expanding stent 358 as described with respect to FIGS. 13A-C. As such, blood is supplied to graft vessel 362 by means of the left ventricle and blood supplying vessel 364. The porous apposition device 366 allows in growth of tissue from both the graft vessel 362 and the target vessel 352 thereby facilitating the supply of blood to target vessel 352 through such tissue in growth and the angiogenic connection established therein.

FIG. 16 illustrates similarly delivered porous substrate device 254 of FIG. 11D having a distally tapering wall defining a lumen therein and having a pointed tip 271 for facilitating penetration into left ventricular wall 280. Coaxially loaded within the lumen of device 254 is a graft vessel 250. The proximal end 267 of graft vessel 250 is affixed to blood-supplying vessel 275 by self-expanding stent 276, as described above. The wall of device 254 extends proximally to overlie target vessel 252. A blood supply is established from a blood supply vessel 275 through graft vessel 250. The pores within device 254 promote in growth of tissue between the appositioned graft vessel 250 and target vessel 252 where the blood supply vessel 275 provides a blood supply to the graft vessel 250 which in turn supplies blood to the target vessel 252.

Kits of the Present Invention

Also provided by the present invention are kits for use in practicing the subject methods. Certain kits of the present invention include at least one subject device of the present invention, as described above. Certain other kits include a plurality of subject devices having various shapes and sizes to accommodate varying vessel sizes or tissue contact surface areas. The subject kits may further include one or more subject systems for delivering and implanting the subject devices as well as accessory materials, such as a bio-adhesive, and/or other instrumentation for performing the subject methods. In certain embodiments, the kits further include one or more types of stimulants and stimulant delivery devices.

The kits may further include instructions for using the subject devices and performing the subject methods. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

It is evident from the above description that the features of the subject methods, devices, systems and kits overcome many of the disadvantages of prior art techniques for forming anastomotic connections between tissue structures. The present invention also provides certain advantages including, but not limited to, the elimination of sutures, staples and other conventional anastomotic devices for creating connections between vessels in the body, the reduction in the amount of skill, precision and accuracy required on behalf of a physician to form connections between vessels, the ability to form such connections between small vessels and tissue structures through minimally invasive incisions and on a beating heart. As such, the subject invention represents a significant contribution to the field of anastomosis and, more generally, to the field of forming connections between tissue structures.

The subject invention is shown and described herein in what is considered to be the most practical, and preferred embodiments. It is recognized, however, that departures may be made there from, which are within the scope of the invention, and that obvious modifications will occur to one skilled in the art upon reading this disclosure.

The specific devices and methods disclosed are considered to be illustrative and not restrictive. Modifications that come within the meaning and range of equivalents of the disclosed concepts, such as those that would readily occur to one skilled in the relevant art, are intended to be included within the scope of the appended claims.

That which is claimed is:

1. A method for facilitating the in situ formation of connections between a graft tissue structure and target tissue structure within the body, comprising the steps of:
accessing said target tissue structure;
providing a graft tissue structure;
positioning said graft tissue structure adjacent said target tissue structure, wherein said graft tissue structure and said target tissue structure contact each other or are in close proximity to each other at at least one selected point of contact or close proximity;
substantially maintaining the position of said graft tissue structure with respect to said target tissue structure while maintaining the tissues of said graft tissue structure and said target tissue structure substantially intact; and
allowing sufficient time for the natural formation of a tissue bond between said graft tissue structure and said target tissue structure at said at least one selected point of contact or close proximity.

2. The method of claim 1 further comprising the step of establishing fluid communication between said graft tissue structure and said target tissue structure.

3. The method of claim 2 wherein said step of establishing fluid communication comprises the step of forming a fluid communication opening at said at least one bonded point of contact or close proximity.

4. The method of claim 3 wherein said fluid communication opening provides a supply of blood to said target tissue structure through said graft tissue structure.

5. The method of 2 wherein said fluid communication is established by the natural vascularization of said tissue bond wherein said vascularization provides a sufficient supply of blood to said target tissue structure.

6. The method of claim 1 further comprising the step of establishing a blood supply to said graft tissue structure.

7. The method of claim 6 wherein said step of establishing a blood supply to said graft tissue structure is performed prior to said step of allowing sufficient time.

8. The method of claim 6 wherein said step of establishing a blood supply to said graft tissue structure is performed after said step of allowing sufficient time.

9. The method of claim 6 wherein said step of establishing a blood supply to said graft tissue structure comprises connecting said graft tissue structure to a blood supply within the body.

10. The method of claim 1 wherein said sufficient time is from about 1 to 180 days.

11. The method of claim 1 or 5 wherein said natural formation or vascularization comprises angiogenic and/or arteriogenic processes.

12. The method of claim 11 further comprising delivering stimulants to said at least one point of contact or close proximity to facilitate said angiogenic and/or arteriogenic processes.

13. The method of claim 1 further comprising disrupting the tissue of at least one of said graft tissue structure and said target tissue structure at said at least one point of contact or close proximity.

14. The method of claim 13 wherein said step of disrupting is performed through a surgical incision.

15. The method of claim 14 wherein said surgical incision is formed by endovascular intervention.

16. The method of claim 15 wherein said endovascular intervention comprises delivering a catheter through said graft tissue structure or through said target tissue structure to said at least one bonded point of contact or close proximity.

17. The method of claim 13 wherein said step of disrupting comprises the step of incising said bonded tissue.

18. The method of claim 13 wherein said step of disrupting comprises removing at least one cell layer of epithelium.

19. The method of claim 18 wherein said step of removing comprises using one or more of the group consisting of laser, thermal, chemical and mechanical means.

20. The method of claim 18 wherein said step of removing is performed endovascularly.

21. The method of claim 1 wherein said step of substantially maintaining said graft tissue structure comprises the step of employing a device configured for holding said graft tissue structure and said target tissue structure in contact or in close proximity at said at least one point of contact or close proximity.

22. The method of claim 21 wherein said device is at least partially biodegradable.

23. The method of claim 21 wherein said device comprises a porous substrate material for facilitating the formation of said at least one tissue bond.

24. The method of claim 21 wherein at least one of said graft tissue structure and said target tissue structure is a vessel and wherein said device is configured to hold a length of said vessel.

25. The method of claim 24 wherein said device has a luminal configuration for holding said length of said vessel.

26. The method of claim 21 wherein at least one of said graft tissue structure and said target tissue structure is a vessel and wherein said device has a configuration for attaching to the distal end of said vessel.

27. The method of claim 1 further comprising the step of delivering an agent to said at least one selected point of contact or close proximity.

28. The method of claim 27 wherein said step of delivering said agent comprises the step of topically applying said agent at said at least one selected point of contact or close proximity.

29. The method of claim 27 wherein said step of delivering said agent comprises the step of injecting said agent into said at least one selected point of contact or close proximity.

30. The method of claim 27 wherein said step of delivering said agent comprises the step of imbedding, seeding or coating said graft tissue structure.

31. The method of claim 27 wherein said step of delivering said agent comprises the step of implanting a device at said at least one point of contact wherein said device comprises or releases an agent.

32. The method of claim 27 wherein said agent is selected from the group consisting of stimulants, gene therapy agents encoding such stimulants and tissue destabilizers.

33. The method of claim 32 wherein said stimulant is a cytokines.

34. The method of claim 32 wherein said gene therapy agent is delivered by means of one or more of the group consisting of a plasmid, a viral vector, a liposomal delivery system and encapsulation into a protein delivery system.

35. The method of claim 1 wherein said graft tissue structure is a vessel.

36. The method of claim 35 wherein said vessel is an autologous vessel.

37. The method of claim 36 wherein said autologous vessel is a pedicled artery.

38. The method of claim 36 wherein said autologous vessel is a section of free graft.

39. The method of claim 36 wherein said autologous vessel is left intact with its native tissue bed.

40. The method of claim 35 wherein said vessel is a homograft or xenograft harvested from a donor.

41. The method of claim 35 wherein said vessel is a pre-fabricated vessel.

42. The method of claim 35 wherein said vessel is formed in situ during said sufficient time.

43. The method of claim 42 wherein said vessel is formed by implanting a scaffolding adjacent said target tissue structure, wherein said scaffolding comprises properties to form a vessel thereabout.

44. The method of claim 6 wherein said blood supply is from the aorta.

45. The method of claim 6 wherein said blood supply is from the left ventricle.

46. A device for facilitating the in situ formation of naturally formed vascularized tissue bonds between a graft tissue structure and at least one target tissue structure within the body without disrupting the tissues of said tissue structures, comprising:
a first surface configured to contact said graft tissue structure;
a second surface configured to contact said target tissue structure; and
at least one opening between said first and second surfaces wherein said graft tissue structure and said target tissue structure are in contact or close proximity through said at least one opening.

47. The device of claim 46 further comprising a means for maintaining said graft tissue structure and said target tissue structure in contact or close proximity.

48. The device of claim 47 wherein said means for maintaining comprises at least one pair of side walls extending from said structure.

49. The device of claim 48 wherein said means for maintaining comprises at least two pairs of side walls extending from said structure.

50. The device of claim 46 wherein at least one of said first surface and said second surface comprises a luminal configuration.

51. The device of claim 48 wherein said at least one pair of side walls is flexible wherein said side walls can be spread apart from each other.

52. The device of claim 48 wherein each of said pairs of side walls is flexible wherein said side walls can be compressed towards each other.

53. The device of claim 46 wherein said means for maintaining comprises an adhesive material.

54. The device of claim 46 wherein said means for maintaining comprises at least one strap or band for positioning around a tissue structure.

55. The device of claim 46 wherein said means for maintaining comprise at least one magnetic means.

56. The device of claim 55 wherein said means for maintaining comprises two magnetic means.

57. The device of claim 46 wherein said structure comprises a docking port for the delivery of a means for creating a fluid communication opening between said graft tissue structure and said target tissue structure.

58. The device of claim 57 wherein said docking port comprises means from the group consisting of magnetic means, electrostatic means and mechanical means.

59. The device of claim 46 wherein said structure includes at least one radio-opaque marker.

60. The device of claim 46 further comprising at least one agent releasable from said structure to promote the natural formation of a vascularized tissue bond between said graft tissue structure and said target tissue structure.

61. The device of claim 60 wherein said structure is made of a biodegradable material, wherein upon biodegradation of said structure, said at least one agent is released from said structure.

62. The device of claim 60 wherein said at least one agent is coated on at least one surface of said structure.

63. The device of claim 60 wherein said agent comprises a tissue growth stimulant or a tissue destabilizer.

64. A device for facilitating the in situ formation of naturally formed vascularized tissue bonds between a first tissue structure and a second tissue structure within the body, comprising:
a structure for appositioning a surface of said first tissue structure with a surface of said second tissue structure at least one point of contact or close proximity;
wherein said structure is made at least in part of a porous material to facilitate the in growth of tissue between said first and said second tissue structures.

65. The device of claim 64 wherein said first tissue structure is pre-attached to said structure.

66. The device of claim 64 wherein said structure comprises a tubular portion.

67. The device of claim 65 wherein said tubular portion is made of said porous material.

68. The device of claim 66 or 67 wherein said tubular portion is configured to hold at least a portion of said first tissue structure.

69. The device of claim 64 wherein said structure is made entirely of said porous material.

70. The device of claim 64 wherein said structure comprises a sharp tip configuration.

71. The device of claim 70 wherein a tubular member extends from a proximal end of said sharp tip.

72. A device for facilitating the in situ formation of naturally formed vascularized tissue bonds between a graft tissue structure and at least one target tissue structure within the body without disrupting the tissues of said tissue structures, comprising:
a cylindrical structure defining a lumen therein having a diameter sufficient to accommodate said graft tissue structure and said at least one target tissue structure in contact or close proximity within said lumen.

73. A system for delivering a graft tissue structure to a target tissue structure within the body and establishing at least one point of contact or close proximity between said graft tissue structure and said target tissue structure, comprising:
a device according to claim 64 wherein said device is attachable to a first end of said graft tissue structure; and
a catheter having at least one lumen configured for receiving said device.

74. The system of claim 73 further comprising an anchoring means attachable to a second end of said graft tissue structure.

75. The system of claim 74 wherein said catheter comprises:
an opening within a wall of said catheter sized to accommodate the passage of said device there through;
an expandable occlusion member on a proximal side of said opening; and
an expandable occlusion member on a distal side of said opening.

76. The system of claim 73 further comprising tissue penetrating means deliverable through said catheter.

77. A kit for facilitating the in situ formation of naturally formed vascularized tissue bonds between a first tissue structure and a second tissue structure within the body, comprising:
at least one device of claim 46;

78. The kit of claim 77 further comprising instructions for using said at least one device.

79. A kit for facilitating the in situ formation of naturally formed vascularized tissue bonds between a first tissue structure and a second tissue structure within the body, comprising:
at least one device of claim 64; and
the system of claim 73;

80. The kit of claim 79 further comprising instructions for using said at least one device.
